(19) United States
(12) Patent Application Publication
(10) Pub. No.: US 2022/0351839 A1
(43) Pub. Date: Nov. 3, 2022

(54) METHODS AND SYSTEM FOR GENERATING AND STRUCTURING MEDICAL EXAMINATION INFORMATION

(71) Applicant: Siemens Healthcare GmbH, Erlangen (DE)

(72) Inventors: Sarah SEIDNITZER, Fuerth (DE); Kathrin EL NEMER, Erlangen (DE); Karlheinz GLASER-SEIDNITZER, Fuerth (DE); Michael RUSITSKA, Utternreuth (DE)

(73) Assignee: Siemens Healthcare GmbH, Erlangen (DE)

(21) Appl. No.: 17/728,153

(22) Filed: Apr. 25, 2022

(30) Foreign Application Priority Data
Apr. 28, 2021 (DE) 10 2021 204 238.4

(51) Int. Cl. G16H 30/40; G16H 15/00; G06T 7/73; G06T 7/10; G06F 3/04842; G06F 3/0486

(52) U.S. Cl. CPC G16H 30/40; G16H 15/00; G06T 7/75; G06T 7/10; G06F 3/04842; G06F 3/0486

(57) ABSTRACT

Computer-implemented methods and devices for structuring or ascertaining at least one piece of medical examination information are provided. Embodiments implement receiving patient data assigned to a patient, building a schematic body model of the patient based on the patient data, wherein the schematic body model schematically replicates at least one anatomy of the patient, identifying at least one piece of examination information in the patient data, determining an anatomical position for the at least one piece of examination information within the schematic body model, generating a visualization of the schematic body model in which the anatomical position of the at least one piece of examination information is highlighted, and displaying the visualization for a user via a user interface.

### CROSS-REFERENCE TO RELATED APPLICATIONS

The present application hereby claims priority under 35 U.S.C. § 119 to German patent application number DE 102021204238.4 filed Apr. 28, 2021, the entire contents of each of which are hereby incorporated herein by reference.

### FIELD

At least some example embodiments relate to the fields of health technology and health informatics and relate to computer-aided or computer-implemented handling of medical examination information. Examination information may in this context be information that is derived from an examination of the patient. This may comprise both measured values or measured datasets, such as medical image data or laboratory data, and clinical findings produced by a physician. At least some example embodiments also relate both to the structured editing of already existing examination information specific to a patient and to the generation of new examination information.

### BACKGROUND

One area of application amongst others lies in the field of medical information systems. Examples of these are RIS information systems (RIS is an abbreviation for the term “Radiology Information System”), hospital information systems (HIS), laboratory information systems (LIS) or the PACS information system (PACS is an abbreviation for the term “Picture Archiving and Communication System”) or combinations thereof. For the targeted diagnostic appraisal of a patient, the user (e.g. a physician, such as e.g. a radiologist, a pathologist or some other clinician) must select or filter out the information relevant to the respective diagnostic assessment task from the available information pertaining to the patient. If e.g. a bone fracture is to be investigated, prior findings relating to lung disease or digital pathological image data are likely to be irrelevant for the diagnostic assessment task. However, due to the volume of available information and the distributed storage of data in the majority of medical information systems, the user is often unable to form any causal overview. In real-world practice it frequently seems that the user is required to load and open different datasets in order to briefly review their contents and decide whether the information contained therein is relevant to the diagnostic assessment task or not. On the other hand, the user is faced with the challenge of taking all relevant information into consideration. Thus, a significant prior examination or prior findings may be highly relevant to the diagnostic assessment task. To remain with the same example, it may be relevant in the diagnostic assessment of a bone fracture whether the bone was already broken or whether other orthopedic limitations of the patient are present.

In addition to the already above-mentioned cognitive effort demanded of the user when combining numerous individual pieces of examination information to arrive at a coherent set of symptoms or a diagnosis, the user must also constantly switch between different types of information representation. Thus, medical image data is represented in the form of individual images, whereas laboratory data is represented in the form of graphs or measured values and prior findings are represented as text. This makes it all the more difficult for the user to obtain the necessary overview in order to acquire a sound understanding of the overall set of symptoms.

A further factor is that in practice the diagnostic assessment tasks for a user are subject to very tight time constraints, which means that in practice little time remains for selecting the examination information relevant to a diagnostic assessment task. The user has just as little time for inputting new examination information. In order to be able to use examination information meaningfully for downstream processes, certain standardized basic information is necessary, relating for example to the position of a clinical finding in the body of the patient or to fundamental characteristics of the finding. If such a structured input is omitted, the value of a piece of examination information in the clinical workflow may be limited.

A further problem is that there are currently serious limitations with regard to automating both the selection and the generation of relevant examination information. One reason for this is that there is often little consistency in the data from patient to patient, as a consequence of which it is very difficult to train corresponding models. Another is that the margin for error in the diagnostic assessment is very small. It is clear, at least at the priority date of the present application, that human decision-making authority is not liable to be replaced by automated processes.

### SUMMARY

Example embodiments provide methods and systems by which examination information relevant to a diagnostic assessment of a patient can be structured in order thereby to improve accessibility to the examination information relevant to a diagnostic assessment task and the possibilities for inputting new examination information.

At least one example embodiment provides a computer-implemented method for structuring medical examination information relating to a patient, the method comprising receiving patient data assigned to the patient; building a schematic body model of the patient based on the patient data, wherein the schematic body model schematically replicates at least one anatomy of the patient; identifying at least one piece of the examination information in the patient data; determining an anatomical position for the at least one piece of the examination information within the schematic body model; generating a visualization of the schematic body model in which the anatomical position of the at least one piece of examination information is highlighted; and displaying the visualization for a user via a user interface.

In at least one example embodiment, the schematic body model is further subdivided into multiple segments, and the determining the anatomical position includes assigning the at least one piece of examination information to a segment.

In at least one example embodiment, the body model is embodied such that each segment is assigned a unique marker, and the determining the anatomical position determines the anatomical position by identifying at least one of the unique markers for the at least one piece of examination information, the assigning being based on the identifying the at least one of the unique markers.

In at least one example embodiment, the unique markers are based on a predetermined anatomical ontology.

In at least one example embodiment, the method further includes identifying at least one relevance segment from the segments of the body model based on at least one of the patient data, the at least one piece of examination information or a diagnostic assessment task, and the generating the visualization includes at least one of highlighting the at least one relevance segment or limiting the visualization to the at least one relevance segment.

In at least one example embodiment, the method further includes identifying at least one further piece of examination information in the patient data based on the relevance segment, and displaying the at least one further piece of examination information via the user interface.

In at least one example embodiment, a plurality of examination information is identified in the patient data and the method further comprises establishing a prioritization of the plurality of examination information based on at least one of the patient data, the examination information or a diagnostic assessment task, wherein the prioritization is based on a relative relevance of a respective piece of examination information within the plurality of examination information, and the generating the visualization is based on the prioritization.

In at least one example embodiment, the method further includes determining one or more attributes based on the at least one piece of examination information; providing a predetermined number of different pictograms, each pictogram representing different attributes of examination information; and assigning one pictogram from the number of different pictograms to the at least one piece of examination information based on the determined attributes, wherein the generating the visualization includes highlighting the anatomical position of the at least one piece of examination information by the assigned pictogram.

In at least one example embodiment, the method further includes providing a predetermined number of different pictograms, each pictogram representing at least one of different attributes or different attribute combinations of a medical report; displaying at least some of the predetermined different pictograms for the user via the user interface; receiving, via the user interface, a user input which comprises the dragging and dropping of a pictogram selected from the displayed pictograms onto a drop site in the displayed visualization; determining an anatomical position based on the drop site; determining one or more attributes based on the selected pictogram; ascertaining a further piece of examination information based on the determined anatomical position and the one or more determined attributes; and assigning the further piece of examination information to the patient data.

In at least one example embodiment, in which the patient data comprises medical image data which represents an anatomical region of the patient, the method further includes establishing a registration between the medical image data and the schematic body model; generating a second visualization based on the medical image data; displaying the second visualization via the user interface; receiving a user input via the user interface, the user input is directed to a generation of a further piece of examination information based on the second visualization; determining an anatomical position for the further piece of examination information based on the user input and the registration; ascertaining the further piece of examination information based on the determined anatomical position and on the user input; and assigning the further piece of examination information to the patient data.

In at least one example embodiment, the schematic body model comprises a whole-body model of the patient, and the visualization of the schematic body model comprises a schematic whole-body view of the patient.

In at least one example embodiment, the schematic body model includes at least one first level of detail and a second level of detail, wherein the second level of detail is an extract from the first level of detail, the level of detail is selectable, and the generating the visualization generates the visualization of the schematic body model based on the selected level of detail.

In at least one example embodiment, the method further includes automatically selecting a level of detail based on at least one of the patient data, the at least one piece of examination information or a diagnostic assessment task.

In at least one example embodiment, the examination information is associated in each case with at least one time point in the patient trajectory, at least one of one or more time points or one or more time ranges are selectable, and the generating the visualization generates the visualization of the schematic body model based on at least one of the selected time points or time ranges.

In at least one example embodiment, the method further includes automatically selecting the at least one of one or more time points or one or more time ranges based on the at least one of the patient data, the at least one piece of examination information or a diagnostic assessment task.

According to at least one example embodiment, a computer-implemented method for ascertaining examination information during a diagnostic assessment of patient data relating to a patient, the method comprising receiving the patient data relating to the patient; generating a visualization based on the patient data, the visualization representing at least one anatomical region of the patient; displaying the visualization for a user via a user interface; providing a predetermined number of different pictograms, each pictogram representing at least one of different attributes or attribute combinations of a possible medical report of the patient; displaying at least some of the predetermined different pictograms to allow selection, dragging and dropping of individual pictograms by the user via the user interface; receiving a user input, the user input comprises a dragging and dropping of a pictogram selected from the displayed pictograms onto a drop site in the displayed visualization; determining an anatomical position of medical findings based on the drop site; determining one or more attributes of the medical findings based on the selected pictogram; ascertaining the examination information based on the determined anatomical position and the one or more determined attributes; and providing the examination information.

According to at least one example embodiment, the providing the examination information includes at least one of producing a medical report based on the examination information, or storing the examination information in the patient data.

According to at least one example embodiment, the generating the visualization includes building a schematic body model of the patient, the body model schematically replicates at least one anatomy of the patient, and the visualization comprises a visual representation of the schematic body model.

According to at least one example embodiment, the patient data comprises medical image data representing the at least one anatomical region of the patient, and the generating the visualization generates a visualization of the medical image data, the method further comprising building a schematic body model of the patient based on the patient data, the body model schematically replicates at least one anatomy of the patient; and establishing a registration between the medical image data and the schematic body model, wherein the anatomical position is determined based on the registration, and the anatomical position is defined relative to the body model.

According to at least one example embodiment, the generating the visualization further comprises selecting the at least one anatomical region of the patient for the visualization, the visualization represents only the selected anatomical region, and the selection is made based on at least one of the patient data or a diagnostic assessment task.

According to at least one example embodiment, a computer-implemented method for ascertaining examination information during a diagnostic assessment of patient data relating to a patient, comprises receiving the patient data relating to the patient, wherein the patient data comprises medical image data that represents an anatomical region of the patient; building a schematic body model of the patient based on the patient data, the body model schematically replicates at least one anatomy of the patient; establishing a registration between the medical image data and the schematic body model; generating a visualization of the medical image data; displaying the visualization for a user via a user interface; receiving a user input via the user interface, the user input is directed to a generation of examination information on the basis of the visualization; determining an anatomical position for the examination information within the schematic body model based on the user input and the registration; ascertaining the examination information based on the determined anatomical position and on the user input; and providing the examination information.

According to at least one example embodiment, a system for structuring medical examination information relating to a patient comprises an interface; and a controller, the controller is configured to cause the system to receive patient data assigned to the patient via the interface, build a schematic body model of the patient based on the patient data, wherein the schematic body model schematically replicates at least one anatomy of the patient, identify at least one piece of the examination information based on the patient data, determine an anatomical position for the at least one piece of examination information within the schematic body model, and generate a visualization of the schematic body model in which the anatomical position of the at least one piece of examination information is highlighted.

According to at least one example embodiment, a system for ascertaining examination information during the diagnostic assessment of patient data comprises an interface; and a controller, the controller is configured to cause the system to receive the patient data via the interface; generate a visualization based on the patient data and to provide it to a user via the interface, the visualization represents at least one anatomical region of the patient; provide a predetermined number of different pictograms, each pictogram representing at least one of different attributes or attribute combinations of a possible medical report of the patient; provide a user with at least some of the predetermined different pictograms via the interface to allow selection, dragging and dropping of individual pictograms onto the visualization by the user; receive a user input of the user via the interface, the user input comprises the dragging and dropping of a pictogram selected from the displayed pictograms onto a drop site in the visualization; determine an anatomical position based on the drop site; determine one or more attributes based on the selected pictogram; determine examination information based on the determined anatomical position and the one or more determined attributes; and provide the examination information.

According to at least one example embodiment, a system for ascertaining examination information during the diagnostic assessment of patient data comprises an interface; and a controller, wherein the patient data comprises medical image data that represents an anatomical region of the patient, and the controller is configured to cause the system to receive the patient data via the interface, generate a visualization of the medical image data and provide it to a user via the interface, build a schematic body model of the patient based on the patient data, the schematic body model schematically replicates at least one anatomy of the patient, establish a registration between the medical image data and the schematic body model, receive a user input via the user interface, the user input is directed to a generation of the examination information based on the visualization, determine an anatomical position for the examination information within the schematic body model based on the user input and the registration, ascertain the examination information based on the determined anatomical position and on the user input, and provide the examination information.

According to at least one example embodiment, a computer program product which comprises a program, when executed by a programmable computing unit of a system, causes the system to perform at least one method according to an example embodiment.

According to at least one example embodiment, a computer-readable storage medium having readable and executable program sections, when executed by a controller of a system, cause the system to perform at least one method according to an example embodiment.

### BRIEF DESCRIPTION OF THE DRAWINGS

Further special features and advantages of example embodiments will become apparent from the following description of exemplary embodiments explained with reference to schematic drawings. Modifications cited in this connection may be combined with one another in each case in order to implement new embodiments. The same reference signs are used for like features in different figures.

In the figures:

FIG. 1 shows a schematic view of an embodiment of a system for structuring medical examination information or for ascertaining structured examination information during the diagnostic assessment of patient data according to one embodiment,

FIG. 2 shows a flowchart of a method for structuring medical examination information,

FIG. 3 shows a graphical user interface for a method for structuring medical examination information or for providing structured examination information according to one embodiment,

FIG. 4 shows a graphical user interface for a method for structuring medical examination information or for providing structured examination information according to one embodiment,

FIG. 5 shows a flowchart of a method for providing structured examination information according to one embodiment,

FIG. 6 shows a graphical user interface for a method for structuring medical examination information or for providing structured examination information according to one embodiment, and

FIG. 7 shows a flowchart of a method for structuring medical examination information and for providing structured examination information according to one embodiment.

### DETAILED DESCRIPTION

The stated object is achieved according to example embodiments by methods, systems, computer program products or computer-readable storage media according to the main claim and the independent claims. Advantageous developments are disclosed in the dependent claims.

The inventive achievement of the object is described below both in relation to the claimed systems and in relation to the claimed methods. Features, advantages or alternative embodiments/aspects mentioned in this regard are equally to be applied also to the other claimed subject matters, and vice versa. In other words, the object-related claims (which are directed for example to a system) can also be developed by the features that are described or claimed in connection with a method. The corresponding functional features of the method can in this case be embodied by corresponding object-related modules.

According to one aspect, a computer-implemented method for structuring medical examination information relating to a patient is provided. The method comprises a number of steps. A first step is directed to a receiving of patient data assigned to the patient. A further step is directed to a building (or generating) of a schematic body model of the patient based on the patient data, the schematic body model schematically replicating at least one anatomy of the patient. A further step is directed to an identifying of at least one piece of examination information in the patient data. A further step is directed to a determining of an anatomical position for the at least one piece of examination information within the schematic body model. A further step is directed to a generation of a visualization of the schematic body model, in which visualization the anatomical position of the at least one piece of examination information is highlighted. A further step is directed to a displaying of the visualization for a user via a user interface.

The patient data contains the medical data available in relation to the patient. The medical data may comprise both medical image data and non-image data. Image data can relate in this context to medical image data having two or three spatial dimensions. Furthermore, the image data may additionally include a temporal dimension. Medical image data in this case is in particular image data that has been acquired by an imaging modality and can represent in particular a part of the body of the patient. Imaging modalities may in this case comprise for example computed tomography devices, magnetic resonance devices, X-ray devices, ultrasound devices and the like. Image data acquired using such or similar modalities is also referred to as radiological image data.

Furthermore, medical image data may comprise digitized histopathological images representing a correspondingly prepared tissue section of the patient. The image data may further comprise longitudinal data, e.g. in the form of time series or follow-up acquisitions spaced apart in time. Non-image data may comprise, in particular longitudinal, data containing one or more medical values of the patient and/or elements from the disease history of the patient. This may be laboratory data, vital parameters and/or other measured values or prior examinations related to the patient. Furthermore, the non-image data may comprise patient-specific demographic details related, say, to age, gender, lifestyle habits, risk factors, etc. Non-image data may also include one or more prior findings and/or other assessments (e.g. by other, possibly referring physicians). These may be included as findings in the patient data for example in the form of one or more structured or unstructured medical reports.

The patient data may in this case be retrieved from one or more databases. For example, the user can select a diagnostic assessment task or, as the case may be, a patient from a worklist. The user may in this case be for example a female or male physician, such as, for example, a female or male radiologist wanting to produce a medical diagnosis for the patient (in the following the patient is also called the “patient to be diagnosed”). The connected databases can be interrogated for the patient data of the patient based on the selection of the diagnostic assessment task or the patient. An electronic identifier, such as e.g. a patient ID or an access number, may be used for this purpose, for example. Accordingly, the patient data can be received from one or more of the available databases, in each of which at least parts of the patient data are stored. The databases may in this case be for example part of medical information systems, such as e.g. hospital information systems and/or PACS systems and/or laboratory information systems, etc.

According to certain embodiments, the patient data may include very comprehensive information of different types about the state of health of the patient (though, according to other embodiments/aspects, this may also be limited to one data category only, e.g. image data, and in this case in particular radiological data). It may be a task of the user, within the scope of a diagnostic assessment task, to produce a report of medical findings or a medical diagnosis or to draw a conclusion based on the medical dataset.

In order to support the user in this process, one or more pieces of examination information are first identified in the patient data. A piece of examination information may generally be information generated in the course of an (earlier) examination. Alternatively or in addition, the piece of examination information may be information that is potentially relevant to a diagnostic assessment task. A piece of examination information may in this case relate to a location or an (anatomical) position on or in the body of the patient, for example to a part of the body, an anatomy, an organ, and/or a possible, in particular pathological, change etc. in a patient. In addition, a piece of examination information may also relate to a number of such locations or positions. The location or the anatomical position may in this case relate both to an isolated point and to an entire region on or in the body of the patient.

A piece of examination information may be a single, in particular self-contained, dataset contained in the patient data. A piece of examination information may for example comprise medical image data that points to or relates to a part of the body, an anatomy, an organ and/or a possible pathological change, etc. Alternatively or in addition, a piece of examination information may comprise non-image data such as e.g. one or more measured values that are related to a part of the body, an anatomy, an organ and/or a possible pathological change, etc. In particular, a piece of examination information may also relate to a single pathological tissue change, such as e.g. a lesion in the lung, and e.g. specify parameters or characteristics of said tissue change such as e.g. a size, a volume, a position, a position relative to other anatomies, a composition, etc. Furthermore, non-image data may include one or more prior findings and/or other assessments (e.g. by other, possibly referring physicians) which are related to a part of the body, an anatomy, an organ and/or a possible, in particular pathological, change, etc. in the patient.

The (anatomical) position associated with the respective examination information may be stored in the examination information directly, e.g. in the form of metadata. In the case of medical image data, it can thus be specified, e.g. in the header, which organ is shown in the image data, from where the tissue sample on which the histopathological image data is based was taken, etc. Alternatively or in addition, the anatomical position may also be derived indirectly from the examination information or the patient data. If a medical report concerns the liver, for example, then this report and the information referenced therein should be assigned to the liver of the patient.

Identifying examination information in the patient data may basically comprise recognizing or acquiring an individual dataset in the patient data. In addition, identifying examination information may comprise determining a type of the respective dataset.

The anatomical positions associated with the identified examination information are then determined. To that end, metadata of the respective examination information may be read out, for example. Alternatively or in addition, if the examination information contains image data, image recognition algorithms may be used in order e.g. to detect an imaged anatomy and/or medical landmarks. Structured and/or unstructured text data may be searched for keywords e.g. using a computational linguistics algorithm.

The anatomical position may be given for example by two- and/or three-dimensional coordinates in the coordinate system of the body model. Alternatively or in addition, the anatomical position may comprise or mark a subsection of the body model, such as e.g. a part of the body or a segment. In particular, the anatomical position may be related (only) to the body model and according to certain embodiments does not necessarily correspond to a really existing position in the anatomy of the patient. The anatomical position may in this case also be a semantic position or position specification which semantically characterizes or defines a region in the body model (e.g. left lung—inferior lobe—basal segment—lateral). The anatomical positions are therefore at least not necessarily already present but according to certain embodiments may be defined first via the body model.

The determined anatomical positions now permit the examination information to be assigned to a schematic body model of the patient in a spatially resolved manner. The schematic body model may be a whole-body model of the patient or model or comprise only parts of the patient. The schematic body model may in this case be constructed or adapted in particular based on the patient data. According to certain aspects, the method may therefore optionally comprise the step of adapting the body model to fit the respective patient based on the patient data specific to the patient. Thus, a different basic model may be used e.g. for a female patient than for a male patient. Furthermore, a body height, a body weight, information concerning implants etc. may be taken into account (if present in the patient data) in the course of building the body model. Even if it is indicated e.g. by the patient data that a lung has been removed, the latter can be omitted accordingly from the body model. The schematic body model is in particular not an exact body model of the patient but maps the anatomy of the patient in simplified and/or schematic form. In particular, the body model can be a two-dimensional model. Furthermore, the body model can be a three-dimensional model.

The spatially resolved assignment of the examination information to the schematic body model, i.e. the identification of the anatomical position, may be accomplished by a registration, for example.

Based on the schematic body model and the determined anatomical positions, it is then possible to generate a visualization in which the anatomical positions are highlighted. For example, an, in particular two-dimensional, assistance image from which the anatomical positions are visually apparent to the user can be rendered based on the schematic body model and the determined anatomical positions. For this purpose, e.g. (in particular colored) markings or icons can be integrated into the assistance image at the anatomical positions. Alternatively, the anatomical positions can be highlighted by symbols and/or text.

To give the user the opportunity of checking whether or which of the examination information is relevant to the present case and if necessary to initiate further steps, the visualization is displayed to the user via a user interface. The user interface can be embodied accordingly, for example in that it implements a graphical user interface having corresponding display functions. The user can furthermore input a user input relating to the visualization/examination information via the user interface. The user interface can also be embodied accordingly for that purpose, for example in that it implements a graphical user interface having corresponding input functions.

The features interact synergetically in order to provide the user with a quick overview of potentially decision-relevant information for a patient. By identifying the examination information, the user does not need to work through the available data sources personally but receives relevant information that has been selected automatically. Assigning the examination information to an anatomical position results in the examination information being structured in a spatially resolved manner. The user is thus provided automatically with supplementary information with which he or she can more effectively interpret and filter the available information by e.g. ignoring anatomical positions that are irrelevant to a diagnostic assessment task. The registration with a body model or the reference to anatomical positions within the body model in this case permits not only structured and intuitive access to the full context information of a diagnostic assessment task but also ensures that this information is available at all times in its entirety during the subsequent diagnostic assessment. In the interests of a continuation of the human-machine interaction, the user can then, in a next step, target and select that examination information that he or she deems relevant. The user is thus effectively supported during the technical task of analyzing medical data.

In terms of reproducing information, it is specified according to example embodiments what or which information is shown, and not how this information is represented. The extracted information relates to the internal status prevailing in the medical information system (which examination information is present in the system for which issue) and enables the user to operate and control the technical system correctly (i.e. to identify the right examination information). The presence of one or more pieces of examination information in the patient data can in this case change dynamically when e.g. new information about the patient becomes available. This can be taken into account automatically by automatic identification of the examination information.

According to one aspect, the method comprises a receiving and/or determining of a diagnostic assessment task. The diagnostic assessment task relates for example to a formulation of a medical diagnosis for the patient based on the patient data. For example, the diagnostic assessment task may be derived from the patient data if e.g. the electronic medical record includes a corresponding task or points to such a task. Furthermore, the diagnostic assessment task may be derived for example from user information about the user, such as, for example, an electronic worklist of the user. Alternatively or in addition, the diagnostic assessment task may be input into the user interface by the user by a user input.

According to one aspect, the method further includes the step of retrieving the patient data from a medical information system. In particular, the retrieval may comprise retrieving a first part of the patient data from a first data source of the medical information system and retrieving a second part of the patient data from a second data source of the medical information system, the first data source being different or separate from the second data source, and the first part of the patient data being different from the second part of the patient data.

As a result of the automatic retrieval of the patient data, the user automatically receives an overview of the full context data for his or her diagnostic assessment task. The retrieval may be accomplished for example using an electronic identifier, such as e.g. a patient ID, a patient name or an access number which uniquely identifies the patient in the medical information system.

According to one aspect, the method further comprises a step of monitoring the data status of the patient data of the patient for a change in the data status, in which case, in the event of a change in the data status, the step of identifying examination information, the step of determining an anatomical position, the step of generating a visualization, and the step of displaying the visualization are repeated. As a result, an updated visualization is automatically displayed to the user, thereby further improving the user's access to potentially decision-relevant information.

According to one aspect, the method further comprises a step of monitoring the data status of the patient data of the patient for a change in the data status, in which case, in the event of a change in the data status, the step of determining an anatomical position, the step of generating a visualization and the step of displaying the visualization are performed repeatedly. As a result, the user is automatically alerted by the visualization to changes in the data status, thereby further improving the user's access to potentially decision-relevant information.

According to a further aspect, the method further comprises a step of outputting a warning message to the user via the user interface in the event of a change in the data status. The warning message may in this case be embodied as an optical and/or acoustic warning message. By the warning message, the user can be alerted in a targeted manner to changes in the data situation and if applicable to an updated visualization. According to example embodiments, newly added examination information, in particular due to a change in the data status, can in this case be highlighted in the visualization.

According to one aspect, the anatomical position or anatomical positions is or are accentuated in the visualization image by highlighting. According to a further aspect, the highlightings are stored by a function which enables the user to access the associated examination information and have at least part of the associated examination information displayed in particular in the user interface. For example, the highlighting may comprise a link to the respective examination information which can be clicked on by the user. As a result of such an interactive configuration of the visualization, the user obtains particularly easy and quick access to the identified examination information.

According to one aspect, the method further comprises the steps of: producing a synopsis for the identified examination information and displaying the synopsis via the user interface.

The synopsis may in this case contain metadata which is derived from the examination information and/or the patient data for the purpose of producing the synopsis. The synopsis may for example comprise one or more attributes, which attributes characterize the examination information. The attributes may for example comprise a creation time of the examination information (e.g. the date of a findings report or of an acquisition of medical image data), a type of the examination information (e.g. whether it concerns radiological images, a medical report or laboratory data), a brief summary of the examination information (e.g. the naming of the diagnosis or a chosen therapy) and/or further information (e.g. a link to an electronic medical compendium or a link to an electronic medical guideline) and further aspects. The synopsis may in particular be generated automatically, in particular by filling a predefined mask with patient data and further data relating to the examination information. In addition, attributes can be extracted from the patient data and/or the examination information by extraction algorithms. For example, computational linguistics algorithms can be used for extracting relevant data from structured or unstructured text documents.

According to certain aspects, the synopsis may be displayed together with and in particular within the visualization. The synopsis may in this case be displayed in particular in such a way that it can be assigned to the respective examination information and in particular to the highlighting. Furthermore, the synopsis can be displayed in response to a receiving of a corresponding user input by the user into the user interface. The user input may in this case comprise for example a clicking on the examination information or its highlighting or a moving of an input element such as a mouse pointer over the examination information or the highlighting.

By the synopsis, the user is automatically provided with relevant information which helps him or her to decide which examination information should be taken into account for the diagnostic assessment task.

According to a further aspect, the visualization is embodied as an interactive visualization in which the user is able to change the display or presentation by a user input. Accordingly, the method comprises a receiving of a user input directed to an adapting of the visualization via the user interface as well as to the adapting of the visualization based on the user input and a displaying of the adapted visualization. According to example embodiments, the adapting comprises an adjusting of the view of the body model and in particular a rotation, shifting, maximizing and/or minimizing the view of the body model. According to further embodiments, the adapting comprises the selection of a (sub-) region of the body model for display.

As a result of this interactive visualization, the user is able to focus on that part of the body (and the associated examination information) which the user requires for the respective diagnostic assessment task.

According to one aspect, the method comprises an ascertaining of at least one piece of examination information from the patient data, in particular by applying a correspondingly embodied application to the patient data. According to one aspect, the patient data comprises radiological image data and the at least one piece of examination information comprises information relating to a pathological change in the patient which is imaged in the radiological image data. According to one aspect, the application for automated determination and/or characterization of the pathological change from the radiological image data is embodied using image data evaluation means, wherein a characterization may in particular comprise the determining of one or more attributes (described in more detail hereinbelow). According to one aspect, the pathological change comprises a morbid tissue change and in particular a tumor. According to one aspect, the pathological change comprises a pathological vascular change and in particular a vascular constriction. According to one aspect, the tissue is in particular lung tissue and the radiological image data shows a lung tissue of the patient. According to one aspect, the vessel is in particular part of a blood vessel system of the patient and the radiological image data shows a part of a blood vessel system of the patient. According to one aspect, the application is embodied to determine pathological tissue changes in radiological image data of lung tissue. According to one aspect, the application is embodied to determine pathological vascular changes in radiological image data of blood vessels.

According to one aspect, the schematic body model is subdivided into multiple segments, and in the step of determining the anatomical position the at least one piece of examination information is assigned to a segment.

The use of segments (which may also be referred to as anatomical segments) simplifies the assignment of the examination information because an exact position in the body model does not necessarily need to be determined, the association with a segment being sufficient. A segment may for example be a part of the body of a patient, such as e.g. a thoracic region, or an organ, such as e.g. the lung, and/or an anatomy of a patient, such as e.g. the ribcage or the spinal column, and/or an arbitrary subregion of the above.

According to one aspect, the schematic body model may be subdivided into a number of segments hierarchically linked to one another.

“Hierarchically linked to one another” can mean in this context that the body model contains a hierarchy of segments. In other words, there are different hierarchical levels (or classes of segments), which hierarchical levels can provide a subdivision into different degrees of detail for an anatomical region of the body model. For example, one hierarchical level may relate to the higher-ranking organs, such as e.g. the lung or brain of a patient. A segment of said hierarchical level would then correspond for example to the respective organ. A further, in particular lower-ranking, hierarchical level may then relate to a subdivision in the respective organ (e.g. right lung vs. left lung). Based thereon, further, even more detailed hierarchical levels may be defined which successively create an ever-finer subdivision and so enable the location or anatomical position to be specified with ever greater accuracy. A segment of a lower-ranking hierarchical level may in this case correspond in particular to a subregion of a segment of the higher-ranking hierarchical level, which segment in turn corresponds to a subregion of a further higher-ranking segment of a further hierarchical level ranking thereabove, etc. The segments of the lower-ranking hierarchical level are then connected hierarchically in this way to the segments of the higher-ranking hierarchical level(s). The number of hierarchical levels is in this case limited to an extent which is just as little as the number of subsegments that are defined in the transition from a higher-ranking hierarchical level to a lower-ranking hierarchical level. In particular, the number of hierarchical levels and the number of respective subsegments may also vary from organ to organ. For example, a very fine classification may be provided for large organs, such as e.g. the lung. In the above example, an anatomical position in the lung was specified by way of example by the sequence of terms or markers as follows: lung—left lung—inferior lobe—basal segment—lateral. Six hierarchical levels are therefore present here. However, fewer or further levels may also be provided according to need and organ, e.g. 2, 3, 4, 5, 7, 8, 9, 10, 11, 12 or more hierarchical levels.

Providing a body model with segments hierarchically linked to one another enables the anatomical position to be precisely localized by identifying the corresponding segment without the user being actively required to specify a position. The anatomical position may in this case be in particular a semantic position which designates one or more segments in a low-ranking (in particular the respective lowest-ranking) hierarchical level.

According to one aspect, the segments, and in particular the hierarchically interlinked segments, are based on a predetermined, in particular standardized, anatomical ontology. In particular, the segments, and in particular the hierarchically interlinked segments, may be created or defined in the body model based on or according to the predetermined anatomical ontology. In other words, the body model may then be constructed or generated based in addition on the predetermined, in particular standardized, anatomical ontology. In other words, the body model is encoded based on or according to the predetermined, in particular standardized, anatomical ontology.

This has the advantage that the results are transferable between different, patient-specific body models and/or between different time points and/or between different medical institutions (for instance: different hospitals or medical disciplines) and in particular that the assignment of the examination information can be simplified.

According to one aspect, the predetermined anatomical ontology is based on the “SNOMED Clinical Terms” terminology or ontology and/or on the “RadLex” terminology or ontology. The use of these per se known standardized terminologies for defining or building the body model ensures the compatibility of the proposed methods with the clinical processes, improves the exchange of information and facilitates the assignment of examination information to the body model.

According to one aspect, each segment is assigned a unique marker, and the anatomical position is determined in the determination step in that at least one of the unique markers is identified for the at least one piece of examination information, as a result of which the at least one piece of examination information is assigned to at least one corresponding segment.

The marker may also be understood as a tag, token or abbreviation. Identifying one or more of the markers for a piece of examination information may in particular comprise an identification based on the examination information itself. Identifying one or more of the markers for a piece of examination information may in particular comprise a search for one or more markers in the examination information. Thus, markers of said type may be contained for example directly in the metadata of a piece of examination information, such as e.g. in a DICOM header, or in the text of a medical report. Furthermore, identifying one or more markers may comprise a mapping of the respective examination information to the marker system. To that end, e.g. computer-implemented functions may be adapted in such a way that they can assign one or more markers to e.g. image or laboratory data. Thus, for example, a function may be trained to assign image data to a marker and consequently to the corresponding segment in the body model. In the case of examination information containing text information, e.g. a “tokenization” may be carried out in which the terms are replaced by the corresponding markers (either according to a rule-based principle or by a trained function). In this case, methods based in particular on word embeddings may be used in which the meaning of a word is encoded into a descriptor (typically in the form of a vector) in such a way that words of similar content lie closer to one another in the results space and can be assigned in a unifying manner to a marker. If reference is made in a medical report e.g. to a “shop window width”, this may indicate a lumbar spinal stenosis. Accordingly, it is necessary here to identify a marker which corresponds to a corresponding segment in the body model.

The use of standardized markers simplifies the assignment of the examination information since the body model is encoded according to these unique markers. This goes beyond a mere naming or labeling of segments in the body model since the body model is systematically constructed in such a way that it corresponds to the unique markers. In other words, the body model is therefore embodied in such a way that each segment of the body model is assigned or can be assigned a unique marker.

The assignment of markers to the segments of the body model is in this case compatible in particular with the hierarchically interlinked segments. The body model is then embodied in such a way that multiple hierarchically interlinked segments are defined in the body model in such a way that they are assigned or assignable in each case to an, in particular predetermined, unique marker.

According to one aspect, the unique markers are based on a predetermined anatomical ontology. Accordingly, rather than an ontology being created or adapted for the body model, the body model is encoded according to an existing ontology, which improves the compatibility with existing clinical processes.

According to one aspect, the predetermined anatomical ontology is based on the “SNOMED Clinical Terms” terminology or ontology and/or on the “RadLex” terminology or ontology. The use of these per se known standardized terminologies for defining the body model ensures the compatibility of the proposed methods with clinical processes, improves the exchange of information and facilitates the assignment of examination information to the body model.

According to one aspect, a plurality of examination information is identified in the patient data and the method comprises establishing a prioritization of the plurality of examination information based on the patient data and/or the examination information, the prioritization being based on a relative relevance of a respective piece of examination information within the plurality of examination information, wherein the step of generating the visualization, and in particular the highlighting of the respective anatomical position, is performed taking the prioritization into account. According to a further aspect, the prioritization is additionally based on the diagnostic assessment task.

By the prioritization, the user can be guided automatically to that examination information which is particularly relevant. This may be for example examination information which enables major discrepancies with regard to previous examination information (in particular within the same segment) to be recognized. For example, a piece of examination information may indicate a clear improvement in a pathological change. Furthermore, an increased relevance of the respective examination information to the diagnostic assessment task may be inferred e.g. on the basis of a semantic or rule-based agreement of elements of a piece of examination information with the diagnostic assessment task. As a result of the prioritization, the user is directed in a targeted manner to relevant examination information and so is effectively supported in the structuring of the examination information e.g. in order to produce a medical diagnosis. The prioritization may be highlighted in the visualization for example using color or by suitable symbols. Furthermore, the visualization may be limited to relevant examination information in the display. In other words, less relevant examination information, whose relevance lies below a predetermined threshold based on the prioritization, may be omitted from the visualization.

According to one aspect, the method further comprises a step of identifying at least one relevance segment from the segments of the body model, in particular based on the patient data and/or the examination information and/or the prioritization and/or the diagnostic assessment task, the at least one relevance segment being highlighted in the step of generating the visualization and/or the visualization being limited to the at least one relevance segment.

This enables the user to be steered in a targeted manner to that part of the body potentially containing relevant examination information. A relevance segment may be highlighted for example in color, by increasing the size of the relevance segment, by focusing on the relevance segment (while non-relevance segments are out of focus and so appear blurred), etc.

According to one aspect, the method further comprises a step of identifying at least one further piece of examination information in the patient data based on the relevance segment, and a displaying of the at least one further piece of examination information via the user interface.

In other words, a targeted search through the patient data is conducted for further information that may be relevant for a user in relation to the relevance segment. Thus, all the potentially relevant information that may be of interest for a diagnostic assessment of the relevance segment is automatically displayed to the user. In particular, this may also include such information that is not necessarily assigned to the anatomical location of the relevance segment. If e.g. a therapy decision is to be taken in relation to a pulmonary nodule, relevant laboratory and/or vital-signs data can be identified and brought to the attention of the user. The further examination information may be identified for example with the aid of the aforementioned unique markers, via which e.g. findings in a pathology report may be linked with a suspicious tissue change in a region of the lung.

According to one aspect, the method further comprises a step of determining one or more attributes based on the at least one piece of examination information, which attributes in each case specify in particular properties of the examination information or characterize the examination information, a step of providing a predetermined number of different pictograms, each representing different attributes of examination information, and a step of assigning one pictogram from the number of different pictograms to the at least one piece of examination information based on the determined attributes, wherein in the step of generating the visualization, the anatomical position of the at least one piece of examination information is highlighted by the assigned pictogram.

A pictogram may in this case represent one or more different attributes. The attributes may relate e.g. to a type of examination information and for example specify whether the examination information contains radiological image data, pathological image data, laboratory data, etc. and/or whether e.g. MR image data, CT image data, ultrasound image data, PET image data, X-ray image data, etc. are contained within the radiological image data. Furthermore, the attributes may relate to a nature of a pathological change. Using a lesion or tumor as an example, the attributes may relate to a contour, blood lipids, a calcium score and/or a solidity score. In particular, the pictograms may be chosen such that they represent parameter ranges of the attributes, using a solidity score as an example, e.g. the parameter ranges non-solid, part-solid and solid. By assigning the pictograms based on the attributes extracted from the examination information, the user receives a structured overview of the available examination information without having to “delve into” the examination information in detail. The user saves time when viewing and filtering the available information and as a result is effectively supported during a diagnostic assessment task.

According to one aspect, the method further comprises the steps of:

providing a predetermined number of different pictograms, each representing different attributes and/or attribute combinations of a medical report; displaying at least some of the predetermined different pictograms to allow selection, dragging and dropping of individual pictograms by the user via the user interface; receiving a user input which comprises the dragging and dropping of a pictogram selected from the displayed pictograms onto a drop site in the displayed visualization; determining an anatomical position within the schematic body model based on the drop site; determining one or more attributes based on the selected pictogram;
ascertaining further examination information based on the determined anatomical position and the one or more determined attributes; and assigning the further examination information to the patient data.

In other words, according to the aspect, a user input by “drag and drop” is supported. By selecting and dragging a pictogram (another word for this is icon), the user can simultaneously specify an anatomical position and attributes for a diagnostic assessment. This greatly simplifies the structured input of findings and thus improves the human-machine interaction. The dropped pictogram can be immediately included in the visualization and displayed permanently in the latter.

If, for example, the user has detected a lesion in the lung tissue and assesses this as spiculated and calcific, he or she can select the pictogram that best reflects these properties and drag it to the location of the lesion in the visualization. Based thereon, corresponding further examination information comprising at least the anatomical position and the pictogram attributes can then be generated automatically. In addition, the further examination information can be assigned further data or information which can be ascertained e.g. from the patient data and/or by an automatic execution of an analysis application to the site within the patient identified by dropping the pictogram. Taking as an example a lesion in the lung tissue identified by dropping, e.g. an analysis application can be applied to associated image data, which analysis application quantifies further measured values, such as e.g. a volume, a contour, an optical characteristic, a change compared to prior examinations, etc.

According to one aspect, the method further comprises a step of identifying at least one further piece of examination information in the patient data based on the anatomical position within the schematic body model determined based on the drop site, and a step of displaying the at least one further piece of examination information via the user interface.

In other words, a targeted search through the patient data is conducted for further information that may be relevant for a user in relation to the anatomical region identified by dragging and dropping. In this way the user automatically receives all the potentially relevant information that may be of significance for the further diagnostic assessment.

According to one aspect, ascertaining the examination information further comprises determining a position related to an anatomy of the patient based on the anatomical position, the examination information being ascertained based in addition on the position related to the anatomy of the patient. In other words, the anatomical position within the body model is converted into a real position, the position related to the patient. The position related to the patient may relate in particular to a medical patient coordinate system which can be spanned for example by medical landmarks of the patient. The position related to the anatomy of the patient may be determined for example based on a registration of the body model with a patient coordinate system.

According to one aspect, the patient data comprises medical image data representing an anatomical region of the patient, and the method further comprises the steps of: establishing a registration between the medical image data and the schematic body model; generating a second visualization based on the medical image data; displaying the second visualization via the user interface; receiving a user input via the user interface, which user input is directed to a generation of further examination information on the basis of the second visualization; determining an anatomical position for the further examination information based on the user input and the registration; ascertaining the further examination information based on the determined anatomical position and on the user input; and assigning the further examination information to the patient data.

The user input may for example comprise an above-described user input by selecting and dragging a pictogram to a drop site—except that the drop site does not lie in a visualization of the schematic body model, but in a visualization of medical image data of the patient. Alternatively or in addition, the user input may comprise an input without the use of pictograms. For example, the user input may comprise a measurement (directly) in the visualization (e.g. of a diameter of a pulmonary nodule) or a selection (directly) in the visualization (e.g. of a potentially relevant region). Accordingly, such information generated by the user input, and in particular measured values or annotations, can be included in the further examination information when the further examination information is being ascertained. The user input may in particular comprise a specification of a location in the visualization based on image data. In other words, it is in particular “spatially resolved” in relation to the visualization. This may be accomplished implicitly, e.g. by clicking on a location in the visualization, or by performing a measurement at a location in the visualization.

A visualization of medical image data may comprise e.g. a two-dimensional representation of the medical image data or a selection from the medical image data. In the case of three-dimensional medical image data, e.g. a section through the image volume can be represented. The additional visualization of the medical image data can be effected alongside the visualization of the schematic body model.

The registration can be e.g. a coordinates-based registration in which coordinates of the schematic body model are registered directly with corresponding coordinates in the medical image data. According to certain aspects, e.g. an elastic registration may be used for this purpose. Alternatively or in addition, the registration may be accomplished based on a semantic registration in which image coordinates are assigned to one or more segments or unique markers of the body model. In particular, the registration may in this case be performed based on a predetermined anatomical ontology.

With the registration, a specification, contained in the user input, of a location in the visualization based on image data can be converted into an anatomical location within the body model. According to the above-described aspect, a spatially resolved input by the user during the diagnostic assessment can therefore be transferred automatically to the body model. The thus defined anatomical region can then be encoded accordingly in the automatically generated further examination information, e.g. in the form of the aforementioned, in particular standardized, predetermined and unique markers, which are advantageously based on a standardized ontology or terminology (e.g. RadLex, SNOMED CT). The user is thus relieved of the work of personally ensuring a correct position specification for his or her diagnostic assessment. Furthermore, the thus obtained examination information can be automatically structured by the systematic mapping to a schematic body model (which is advantageously encoded according to an, in particular standardized, anatomical ontology) (or, as the case may be, likewise encoded according to an, in particular standardized, anatomical ontology) in such a way that it is compatible for further uses.

According to one aspect, the method further comprises a step of identifying at least one further piece of examination information in the patient data based on the anatomical position within the schematic body model determined based on the drop site in the second visualization, and a step of displaying the at least one further piece of examination information via the user interface.

In other words, a targeted search through the patient data is conducted for further information that may be relevant for a user in relation to the anatomical region identified in the second visualization by the user input. Thus, the user automatically receives a display of all the potentially relevant information that may be of significance for the further diagnostic assessment.

According to one aspect, the schematic body model comprises a whole-body model of the patient and the visualization of the schematic body model comprises a schematic whole-body view of the patient.

Using a whole-body model enables all the examination information to be assigned and the user is provided with a holistic picture of the available information pertaining to the patient.

According to one aspect, the schematic body model incudes at least one first level of detail and a second level of detail, wherein the second level of detail is an extract from the first level of detail, wherein the first and/or the second level of detail are/is selectable, in particular by a user input directed to a selection of a level of detail and received via the user input, and in the step of generating the visualization, the schematic body model is visualized based on the selected level of detail.

According to one aspect, the level of detail is selectable by a user input by a user via the user interface and the method further comprises a step of receiving a user input of the user directed to the selection of a level of detail.

For example, the first level of detail may correspond to an overall view of the body of the patient and the second level of detail may correspond to a subregion of the body of the patient, such as e.g. an organ, an anatomy or a part of the body. In particular, the schematic body model may also include multiple different second levels of detail, each corresponding to different subregions of the body of the patient. Alternatively or in addition, the schematic body model may also include even further levels of detail, which further levels of detail are an extract from the second level or levels of detail. For example, a first level of detail may correspond to an overall view of the body of the patient and the second level of detail may correspond to lungs of the patient. A further level of detail may then correspond to a right or left lung of the patient.

According to one aspect, the method further comprises a step of an automatic selection of a level of detail based on the patient data and/or the at least one piece of examination information and/or a diagnostic assessment task.

The automatic selection of the level of detail enables the focus of the user to be guided automatically to that part of the body that is likely relevant to him or her, or, as the case may be, contains the relevant examination information. According to one aspect, the selection may further be based on the aforementioned prioritization. If, for example, the diagnostic assessment of a scan of the lung of a patient is pending as the diagnostic assessment task, a second level of detail corresponding to the lung of the patient can be selected automatically. The attention of the user is thus drawn in a targeted manner to examination information relating to said organ, which further structures the available information for the user.

According to one aspect, the method further comprises a step of identifying at least one piece of further examination information in the patient data based on the selected level of detail, and a step of displaying the at least one piece of further examination information via the user interface.

In other words, a targeted search through the patient data is conducted for further information that may be relevant to a user in relation to the automatically selected level of detail or the level of detail selected by the user. Thus, the user automatically receives a display of all the potentially relevant information that may be of significance for a diagnostic assessment of the relevance segment.

According to one aspect, the examination information is associated in each case with at least one time point (in other words, at least one time point in the patient trajectory may be assigned to each piece of examination information), one or more time points and/or time ranges being selectable, in particular by a user input received via the user interface and directed to a selection of one or more time points and/or time ranges, and in the step of generating the visualization, the schematic body model is visualized based on the selected time points and/or time ranges.

According to one aspect, the one or more time points and/or time ranges are selectable by a user input by a user via the user interface and the method further comprises a step of receiving a user input of the user directed to the selection of the one or more time points and/or time ranges.

Taking the time points into account enables the available information to be better structured and the options for filtering the data are further improved. Thus, this allows a targeted selection of that examination information that is relevant for the user on account of its precedence with respect to time.

According to one aspect, the method comprises an automatic selection of one or more time points and/or time ranges based on the patient data and/or the at least one piece of examination information and/or a diagnostic assessment task. As a result, the user automatically receives a display of that examination information that is relevant with respect to time. The user is thereby supported in the structuring of the available information and consequently in the derivation of a medical diagnosis. According to one aspect, in particular the prioritization can be taken into account in the selection of the one or more time points and/or time ranges.

The selection of one or more time points and/or time ranges may in this case be combined with the selection of a level of detail, as a result of which an even more targeted focusing on relevant examination information can be achieved.

Another example embodiment relates to a computer-implemented method for ascertaining examination information during the diagnostic assessment of patient data. The method comprises a number of steps. One step is directed to a receiving of patient data relating to a patient. A further step is directed to a generation of a visualization based on the patient data, which visualization represents at least one anatomical region of the patient. A further step is directed to a displaying of the visualization for a user via a user interface. A further step is directed to a providing of a predetermined number of different pictograms, each representing different attributes and/or attribute combinations of a possible medical report pertaining to the patient. A further step is directed to a displaying of at least some of the predetermined different pictograms to allow selection, dragging and dropping of individual pictograms by the user via the user interface in order thereby to input a medical report. A further step is directed to a receiving of a user input, which user input comprises the dragging and dropping of a selected pictogram from the displayed pictograms onto a drop site in the displayed visualization. A further step is directed to a determining of an anatomical position based on the drop site. A further step is directed to a determining of one or more attributes of the medical report based on the selected pictogram. A further step is directed to an ascertaining of examination information based on the determined anatomical position and the one or more determined attributes. A further step is directed to a providing of the examination information.

The features of the above-cited aspects may be combined with the preceding aspect provided they are not mutually exclusive. The explanations given with regard to the above-cited aspects and the cited advantages apply analogously to the preceding aspect. In particular, the above-described method may be combined with the above-cited structuring and visualization of already existing examination information.

The preceding aspect represents in a certain sense a reversal of the previous aspects in that it applies the inventive concepts for structuring medical examination information during the input of a medical diagnosis/a medical report and the generation of examination information based thereon. The user is therefore provided not only with a simple and efficient input means, but in addition it is also ensured that the input of the user is systematically realized in such a structured manner that it is suitable for further use in the future (e.g. for a further diagnostic assessment task or in the generation of a medical report).

The visualization may in this case be based on a schematic body model of the patient embodied as described above. Alternatively or in addition, the visualization may also be based on medical image data (e.g. radiological image data or histopathological image data) and comprise e.g. a real image of a part of the body of the patient rendered therefrom, into which image the user can place the pictograms by dragging and dropping.

The anatomical position may in this case be in particular a position defined in respect of the aforementioned body model and in particular a semantic position, e.g. in the form of the unique markers which e.g. can specify or uniquely designate a segment of the body model. A spatially resolved input by the user during the diagnostic assessment can consequently be mapped automatically onto the body model and the thus defined anatomical region can be encoded accordingly in the automatically generated examination information, e.g. in the form of the aforementioned, in particular standardized, predetermined and unique markers, which are advantageously based on a standardized terminology (e.g. RadLex, SNOMED CT).

If the visualization is based on medical image data, the anatomical position may be based for example on a registration of the medical image data with the body model. As already indicated further above, the registration can be e.g. a coordinates-based registration in which model coordinates are registered with corresponding image coordinates. Alternatively or in addition, the registration may be based on a semantic registration in which image coordinates are assigned to one or more segments or unique markers of the body model.

According to one aspect, the patient data accordingly comprises medical image data that represents an anatomical region of the patient, and the step of generating the visualization comprises the generation of a visualization of the medical image data. The method may then further comprise the following steps of: generating a schematic body model of the patient based on the patient data, which body model schematically replicates at least one anatomy of the patient, and establishing a registration between the medical image data and the schematic body model, the anatomical position being determined based on the registration, and the anatomical position being defined in relation to the body model.

The anatomical position is consequently determined automatically for a diagnostic assessment conducted on the basis of image data and furthermore can be transferred into the systematic structure of the body model. This does not make the work of the user any easier but ensures that the thus generated examination information is compatible with downstream clinical processes and with the further clinical data processing. In particular, a search based on the thus established anatomical position can be conducted in the patient data for further examination information which may be relevant to the new examination information generated on the basis of the user input. As already described further above, in particular unique markers assigned within the schematic body model can be resorted to for this purpose.

According to one aspect, the step of ascertaining the examination information further comprises determining a position related to an anatomy of the patient based on the anatomical position, the examination information additionally being ascertained based on the position referred to the anatomy of the patient. In other words, the anatomical position within the visualization is converted into a real position, i.e. the position related to the patient. The position related to the patient may relate in particular to a medical patient coordinate system which can be spanned for example by medical landmarks of the patient. The position related to the anatomy of the patient may be determined for example based on a registration of the visualization with a patient coordinate system.

According to one aspect, the method further comprises a step of identifying at least one further piece of examination information in the patient data based on the anatomical position within the schematic body model determined based on the drop site, and a step of displaying the at least one piece of further examination information via the user interface. In other words, a targeted search through the patient data is conducted for further information that may be relevant for a user in relation to the anatomical position identified by “drag and drop”. The user is thus provided automatically with a display of all the potentially relevant information that may be of significance for the further diagnostic assessment.

According to one aspect, the method may further comprise a step of selecting some of the predetermined number of different pictograms for display, the selection being based on a diagnostic assessment task and/or on already existing examination information and/or on the patient data and/or on an anatomical region of the patient shown in the visualization. As a result, the user can be provided with precisely those pictograms that are likely relevant to the patient/the report to be produced. If e.g. a lung tissue of the patient is to be investigated for lung carcinomas, such pictograms may be selected which characterize a lung carcinoma, e.g. in terms of contour, calcium score, fat content, etc. Alternatively or in addition, the user may also manually select some of the predetermined number or manually modify the automatic selection. Furthermore, all the pictograms of the predetermined number may of course also be displayed.

According to one aspect, the method further comprises a step of producing a medical report based on the examination information, and/or a step of storing the examination information in the patient data.

The possibility of structured data input synergetically benefits the production of an in particular structured medical report, since the automatically extracted information can easily be transferred into a medical report. This further reduces the workload of the user. By storing the examination information, the ascertained examination information is archived and made available for future analyses.

According to one aspect, the step of ascertaining the examination information is further based on the patient data. According to one aspect, the ascertaining of the examination information comprises extracting secondary information, in particular from the patient data, and assigning the secondary information to the ascertained examination information. For example, such secondary information may comprise an ID of the user, a data basis for the findings (e.g. image data based on a CT scan), a date, etc.

According to one aspect, the step of generating the visualization comprises producing a schematic body model of the patient, which body model schematically replicates at least one anatomy of the patient, the visualization comprising a visual representation of the schematic body model.

Because the visualization builds on a schematic body model (which may be embodied as described above), the user can simply assign the pictograms to an anatomical region. In particular, the body model may, as described above, include multiple levels of detail, which can allow a more precise differentiation of the anatomical position.

Alternatively or in addition, the visualization may comprise real image data of the anatomy of the patient, for example radiological or histopathological image data. The anatomical position may then be derived e.g. from the pixel or voxel coordinates of those pixels or voxels of the image data onto which a pictogram has been dropped.

According to one aspect, the step of generating the visualization further comprises selecting an anatomical region of the patient for the visualization, the visualization representing only the selected region and the selection being made based on the patient data and/or information relating to the diagnostic assessment, in particular of a diagnostic assessment task.

The anatomical region may be selected for example by the aforementioned levels of detail. As a result of the selection, the user can be guided in a targeted manner to the relevant anatomy during the diagnostic assessment. This simultaneously reduces the workload of the user and supports him or her in a guided human-machine interaction during his or her diagnostic assessment task.

According to a further aspect, a computer-implemented method for ascertaining examination information during the diagnostic assessment of patient data is provided. The method comprises a number of steps. One step is directed to a receiving of patient data relating to a patient, the patient data comprising medical image data that represents an anatomical region of the patient. A further step is directed to a generation of a schematic body model of the patient based on the patient data, which body model schematically replicates at least one anatomy of the patient. A further step is directed to an establishing of a registration between the medical image data and the schematic body model. A further step is directed to a generation of a visualization of the medical image data. A further step is directed to a displaying of the visualization for a user via a user interface. A further step is directed to a receiving of a user input via the user interface, which user input is directed to an ascertaining of examination information on the basis of the visualization. A further step is directed to a determining of an anatomical position for the examination information within the schematic body model based on the user input and the registration. A further step is directed to an ascertaining of the examination information based on the determined anatomical position and on the user input. A further step is directed to a providing of the examination information.

The features of the above-cited aspects may be combined with the preceding aspect provided they are not mutually exclusive. The explanations given with regard to the above-cited aspects and the cited advantages apply analogously to the preceding aspect. In particular, the above-described method may be combined with the above-cited structuring and visualization of already existing examination information.

The subject matter of the preceding aspect is that a user input relating to an input of examination information must not necessarily be performed in a visualization related to the body model in order to obtain an anatomical position related to the schematic body model and thereby advantageously structure or encode the examination information. Such a relationship is established in the preceding aspect by way of a registration of the schematic body model with the image data.

According to a further aspect, a system for structuring medical examination information relating to a patient is provided. The system comprises an interface and a controller. The controller is embodied to receive patient data assigned to the patient via the interface, to build a schematic body model of the patient based on the patient data, the schematic body model schematically replicating at least one anatomy of the patient, to identify at least one piece of examination information based on the patient data, to determine an anatomical position for the at least one piece of examination information within the schematic body model, and to generate a visualization of the schematic body model in which the anatomical position of the at least one piece of examination information is highlighted.

According to a further aspect, a system for providing examination information during the diagnostic assessment of patient data is provided. The system comprises an interface and a controller. The controller is embodied to receive the patient data via the interface, to generate a visualization based on the patient data and provide it to a user via the interface, which visualization represents at least one anatomical region of the patient, to provide a predetermined number of different pictograms, each representing different attributes and/or attribute combinations of a possible medical report of the patient, to provide a user with at least some of the predetermined different pictograms via the interface to allow selection, dragging and dropping of individual pictograms onto the visualization by the user, to receive a user input of the user via the interface, which user input comprises the dragging and dropping of a pictogram selected from the displayed pictograms onto a drop site in the visualization, to determine an anatomical position based on the drop site, to determine one or more attributes based on the selected pictogram, to determine examination information based on the determined anatomical position and the one or more determined attributes, and to provide the examination information.

According to a further aspect, a system for ascertaining examination information during the diagnostic assessment of patient data is provided, the patient data comprising medical image data representing an anatomical region of the patient. The system comprises an interface and a controller. The controller is embodied to receive the patient data via the interface, to generate a visualization of the medical image data and provide it to a user via the interface, to create a schematic body model of the patient based on the patient data, which schematic body model schematically replicates at least one anatomy of the patient, to establish a registration between the medical image data and the schematic body model, to receive a user input via the user interface, which user input is directed to generating examination information based on the visualization, to determine an anatomical position for the examination information within the schematic body model based on the user input and the registration, to ascertain the examination information based on the determined anatomical position and on the user input, and to provide the examination information.

The controller of the previous aspects may be embodied as a centralized or decentralized computing unit. The computing unit may feature one or more processors. The processors may be embodied as a central processing unit (CPU) and/or as a graphics processing unit (GPU). Alternatively, the controller may be implemented as a local or cloud-based processing server. The controller may furthermore comprise one or more virtual machines.

The interface of the previous aspects may be embodied generally for exchanging data between the controller and further components. The interface may be implemented in the form of one or more individual data interfaces which may include a hardware and/or software interface, e.g. a PCI bus, a USB interface, a FireWire interface, a Zigbee or a Bluetooth interface. The interface may further include an interface of a communications network, in which case the communications network may comprise a local area network (LAN), for example an intranet, or a wide area network (WAN). Accordingly, the one or more data interfaces may include a LAN interface or a wireless LAN interface (WLAN or Wi-Fi). The interface may be further embodied for communicating with the user via a user interface. Accordingly, the controller may be embodied to display the visualization via the user interface and to receive a user input via the user interface.

The advantages of the proposed systems substantially correspond to the advantages of the proposed methods. Features, advantages or alternative embodiments/aspects may equally be applied to the other claimed subject matters, and vice versa.

According to one aspect, the system further includes a data source for storing patient data. The interface maintains a data connection to the data source. The controller is further embodied to access the patient data. The controller is further embodied to select patient data from the data source.

The data source may be embodied as a centralized or decentralized storage unit. The data source may in particular be part of a server system. The data source may in particular be part of a medical information system such as e.g. a hospital information system and/or a PACS system and/or a laboratory information system and/or further information systems of similar type. The data source may be further embodied as cloud storage.

According to a further aspect, a diagnostic assessment system is provided which comprises the system for structuring medical examination information relating to a patient and/or the system for ascertaining examination information during the diagnostic assessment of patient data, as well as a medical information system which is embodied for storing and/or providing patient data. In this case the medical information system is connected via the interface to the system for structuring medical examination information relating to a patient and/or the system for ascertaining examination information during the diagnostic assessment of patient data. The medical information system may furthermore comprise one or more imaging modalities, such as e.g. a computed tomography system, a magnetic resonance system, an angiography system, an X-ray system, a positron-emission tomography system, a mammography system, and/or a system for generating histopathological image data.

At least another example embodiment relates to a computer program product which comprises a program and can be loaded directly into a memory of a programmable controller and has program means, e.g. libraries and help functions, in order to perform a method for providing similarity information, in particular according to the aforementioned embodiments/aspects when the computer program product is executed.

At least another example embodiment to a computer-readable storage medium on which readable and executable program sections are stored in order to perform all the steps of a method for providing similarity information according to the aforementioned embodiments/aspects when the program sections are executed by the controller.

The computer program products may in this case comprise software having a source code which still needs to be compiled and linked or which only needs to be interpreted, or an executable software code which only requires to be loaded into the processing unit in order to execute. The computer program products enable the methods to be performed quickly and in an identically repeatable and robust manner. The computer program products are configured in such a way that they can perform the inventive method steps by the computing unit. The computing unit must in this case fulfill the respective requirements, such as, for example, having a suitable main memory, a suitable processor, a suitable graphics card or a suitable logic unit, so that the respective method steps can be performed efficiently.

The computer program products are stored for example on a computer-readable storage medium or are held resident on a network or server, from where they can be loaded into the processor of the respective computing unit, which processor is connected directly to the computing unit or may be embodied as part of the computing unit. Control information of the computer program products may also be stored on a computer-readable storage medium. The control information of the computer-readable storage medium may be embodied in such a way that it performs an inventive method when the data medium is used in a computing unit. Examples of computer-readable storage media are a DVD, a magnetic tape or a USB stick on which electronically readable control information, in particular software, is stored. When this control information is read from the data medium and loaded into a computing unit, all the inventive embodiments/aspects of the above-described methods can be performed. Thus, example embodiment may also relate to the said computer-readable medium and/or the said computer-readable storage medium. The advantages of the proposed computer program products or the associated computer-readable media substantially correspond to the advantages of the proposed methods.

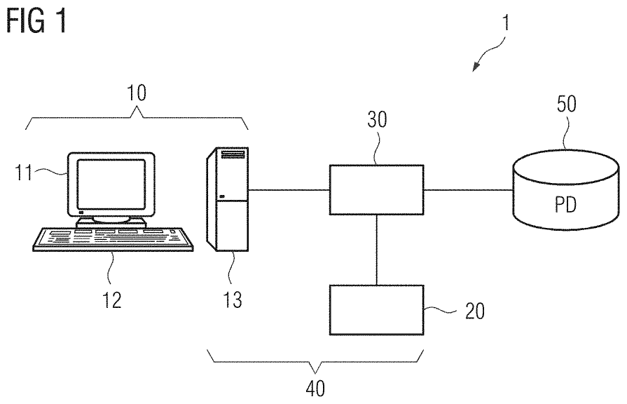

FIG. 1 shows a system 1 for structuring medical examination information or for providing structured examination information based on patient data PD relating to a patient according to one embodiment. The system 1 has a user interface 10, a computing unit 20, an interface 30 and a storage unit 50. The computing unit 20 is basically embodied for structuring medical examination information or for providing structured examination information based on patient data PD. The patient data PD may be provided to the computing unit 20 by the storage unit 50 via the interface 30.

The storage unit 50 may be embodied as a centralized or decentralized database. The storage unit 50 may in particular be part of a server system. The storage unit 50 may in particular be part of a medical information system such as e.g. a hospital information system, a PACS system, a laboratory information system and/or further medical information systems. The storage unit 50 may furthermore be embodied as cloud storage. The storage unit 50 is embodied for storing a quantity of patient data PD. The storage unit 50 may also be referred to as a data source.

A piece of examination information may be a single, in particular self-contained, dataset contained in the patient data PD.

The patient data PD and/or the examination information may include medical image data and/or other medical data comprising no image information. Image data may in this context refer to medical image data having two or three spatial dimensions. Furthermore, the image data may additionally have a temporal dimension. The image data may have been generated for example by a medical imaging modality, such as e.g. an X-ray, computed tomography, magnetic resonance, positron-emission tomography or angiography device or further devices. Such image data may also be referred to as radiological image data.

Furthermore, patient data PD and/or the examination information may also comprise histopathological image data, in each case showing one or more histopathological images. Histopathological image data is image data based on a tissue sample of a patient. Tissue sections are prepared from the tissue sample and stained with a histological dye. The tissue sections prepared in this way are then digitized in order to obtain the histopathological image data. Specialized scanners, also known as slide scanners, may be used for this purpose. The image acquired in the process is also referred to as a “whole slide image”. The image data acquired during this process typically consists of two-dimensional pixel data.

The image data contained in the patient data PD and/or in the examination information may for example be formatted according to the DICOM format. DICOM (=Digital Imaging and Communications in Medicine) is an open standard for the communication and administration of medical image data and associated data.

In addition to image data, the patient data PD and/or the examination information may also comprise non-image data. Non-image data may be e.g. examination results which are not based on medical imaging. This may comprise laboratory data, vital-signs data, spirometry data or the protocols of neurological examinations. Non-image data may additionally comprise text datasets, such as e.g. structured and unstructured medical reports. Furthermore, non-image data may also be patient-related data. This may comprise e.g. demographic details relating to the patient, e.g. relating to his or her age, gender or body weight. The non-image data may be integrated into the image data e.g. as metadata. Alternatively or in addition, the non-image data may also be stored in an electronic medical record (EMR) of the patient, i.e. separately from the image data. Such electronic medical records may be archived for example in the storage device 50 or in a storage device set up separately therefrom, to which the computing unit 20 can be connected via the interface 30.

The user interface 10 may include a display unit 11 and an input unit 12. The user interface 10 may be embodied as a portable computer system, such as e.g. a smartphone, tablet computer or laptop. Further, the user interface 10 may be embodied as a desktop PC. The input unit 12 may be integrated in the display unit 11, for example in the form of a touch-sensitive screen. As an alternative thereto or in addition, the input unit 12 may include a keyboard or a computer mouse and/or a digital stylus. The display unit 11 is embodied to represent, in particular graphically, single or multiple elements from the patient data PD and/or the examination information or a visualization VIS to support interaction with the user. The user interface 10 is further embodied to receive an input from the user relating to an interaction with the system 1.

The user interface 10 includes one or more processors 13 which are embodied to execute a piece of software for controlling the display unit 11 and the input unit 12 in order to provide a graphical user interface GUI which e.g. enables the user to select a patient or the associated patient data PD for a diagnostic assessment task, to recognize or review the visualization VIS or the examination information, to filter or select examination information, to input further examination information, and/or to interactively adapt the visualization VIS. The user can activate the software for example via the user interface 10, for example by downloading it from an app store and/or executing it locally. According to further embodiments, the software may also be a client-server computer program in the form of a web application which runs in a browser.

The interface 30 may have one or more individual data interfaces which ensure the data exchange between the components 10, 20, 50 of the system 1. The one or more data interfaces may be part of the user interface 10, the computing unit 20 and/or the storage unit 50. The one or more data interfaces may include a hardware and/or software interface, e.g. a PCI bus, a USB interface, a FireWire interface, a Zigbee or a Bluetooth interface. The one or more data interfaces may include an interface of a communications network, in which case the communications network may be a local area network (LAN), for example an intranet, or a wide area network (WAN). Accordingly, the one or more data interfaces may include a LAN interface and/or a wireless LAN interface (WLAN or Wi-Fi).

The computing unit 20 may include a processor. The processor may comprise a central processing unit (CPU), a graphics processing unit (GPU), a digital signal processor (DSP), an image processing processor, an integrated (digital or analog) circuit or combinations of the aforementioned components and further devices for structuring medical examination information and/or for providing structured examination information according to example embodiments. The computing unit 20 may be implemented as a single component or comprise a plurality of components operating in parallel or serially. Alternatively, the computing unit 20 may comprise a real or virtual group of computers, such as e.g. a cluster or a cloud. Such a system can be called a server system. Depending on embodiment, the computing unit 20 may be embodied as a local server or a cloud server. Furthermore, the computing unit 20 may include a main memory, such as a RAM, in order for example to temporarily store the patient data PD or the examination information. Alternatively, such a main memory may also be arranged in the user interface 10. The computing unit 20 is embodied e.g. by computer-readable instructions, by design and/or hardware, in such a way that it can perform one or more method steps according to example embodiments.

The computing unit 20 and the processor 13 may together form the controller 40. It should be noted that the illustrated layout of the controller 40, i.e. the depicted division into the computing unit 20 and the processor 13, is likewise to be understood only as by way of example. Thus, the computing unit 20 may be fully integrated in the processor 13 and vice versa. In particular, the method steps may run completely on the processor 13 of the user interface 10 by the execution of a corresponding computer program product (e.g. a software application installed on the user interface), which then interacts directly e.g. with the storage unit 50 via the interface 30. In other words, the computing unit 20 would then be identical with the processor 13.

As already mentioned, the computing unit 20 may, according to certain embodiments, alternatively be understood as a server system, such as e.g. a local server or a cloud server. In such an embodiment, the user interface 10 may be referred to as a “frontend” or “client”, while the computing unit 20 may then be regarded as a “backend”. Communication between the user interface 10 and the computing unit 20 may then be implemented for example based on an https protocol. In such systems, the computing power may be shared between the client and the server. In a “thin client” system the server possesses the majority of the computing power, whereas in a “thick client” system the client provides more computing power. A similar distinction applies to the data (in this case: in particular the patient data PD and the examination information). Whereas in the “thin client” system, the data mostly remains on the server and only the results are forwarded to the client, in the “thick client” system data is also transmitted to the client.

According to further embodiments, the described functionality may also be provided as a cloud service or a web service. The correspondingly embodied computing unit is then realized as a cloud or web platform. The data to be analyzed, i.e. the patient data PD, can then be uploaded to said platform (e.g. via a suitable web interface).

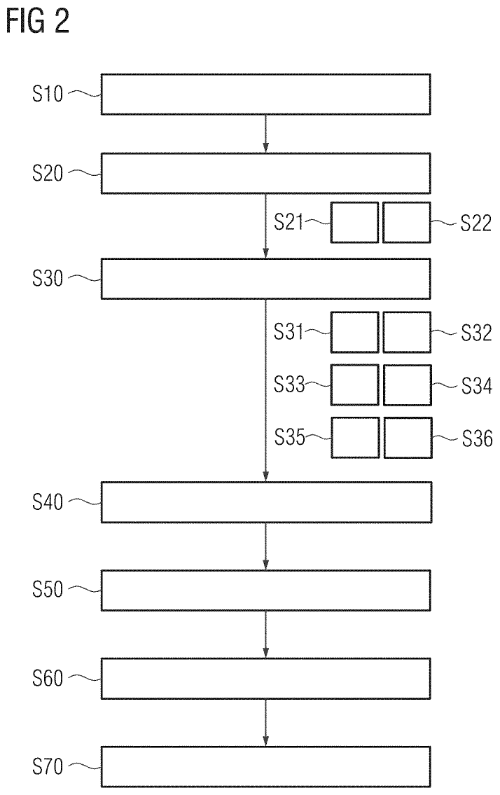

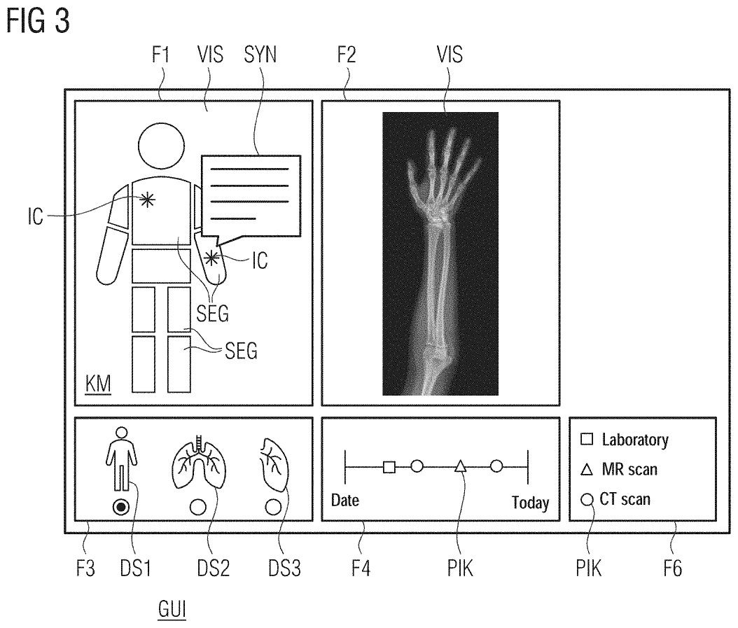

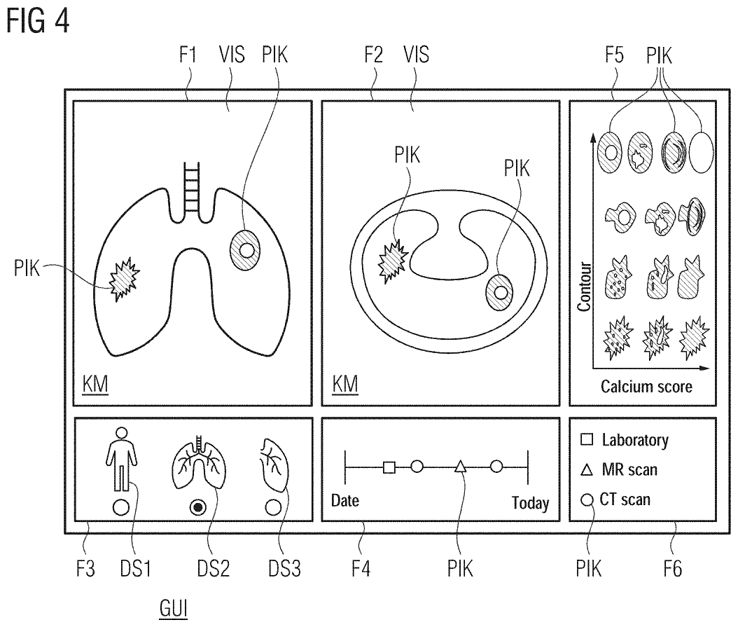

FIG. 2 shows a schematic flowchart of a method for structuring medical examination information relating to a patient. The order of the method steps is limited neither by the illustrated sequence nor by the chosen numbering scheme. Thus, the order of the steps can be swapped if necessary and individual steps can be omitted. Furthermore, one or more steps, in particular a sequence of steps, and optionally the entire method can be performed repeatedly. Associated graphical user interfaces are shown by way of example in FIGS. 3 and 4.

In a first step S10, the patient data PD of the patient to be diagnosed is provided. This may comprise a manual selection of the respective case by a user via the user interface 10, e.g. from a worklist displayed via the user interface 10. This may further comprise a loading of the patient data PD from the data source 50 or another data storage device, which may for example be part of a medical information system. In addition, step S10 may comprise a receiving of the patient data PD by the computing unit 40. Optionally, a diagnostic assessment task may be provided in step S10. A diagnostic assessment task may be understood in this context as a task for the user to derive a specific medical finding from the available patient data PD or to formulate a medical diagnosis. The diagnostic assessment task may be taken in step S10 for example from the worklist and/or extracted from the patient data PD and thus provided. Alternatively or in addition, the diagnostic assessment task may be provided by a corresponding user input into the user interface 10.

In step S20, a schematic body model KM of the patient is constructed. For this purpose, a generic body model KM may be invoked for example based on the patient data PD and adapted based on the patient data PD, e.g. to a height, a gender, a weight or other physiological characteristics of the patient. The body model KM may be a two- or three-dimensional model. One or more segments SEG may be defined in the body model KM, which segments may relate for example to subregions of the anatomy of the patient, e.g. parts of the body (cf. FIG. 3). In an optional substep S21, a relevance segment that is relevant to the diagnostic assessment task may be determined based for example on the patient data PD, the examination information and/or the diagnostic assessment task. If, for example, a humerus fracture is to be diagnosed, the corresponding segment SEG of the body model KM can be identified as the relevance segment. The body model KM may furthermore include different levels of detail DS1, DS2, DS3 (cf. FIG. 3) by which the user can “zoom into” an anatomy. For example, a certain level of detail DS1, DS2, DS3 may relate to the lung of the patient, while a comparatively more general level of detail DS1, DS2, DS3 represents the entire body of the patient, and a comparatively finer level of detail DS1, DS2, DS3 is directed to a right or left lung. As shown in FIGS. 3 and 4, the levels of detail DS1, DS2, DS3 can be selected by a user input. This is illustrated in FIGS. 3 and 4 by way of example by a “radio button” in window F3. Alternatively or in addition, the levels of detail DS1, DS2, DS3 may be created or selected automatically in an optional step S22. This can be implemented based on the patient data PD, the examination information and/or the diagnostic assessment task.

In a step S30, one or more pieces of examination information are identified in the patient data PD. In this case the examination information may be in self-contained parts of the patient data PD which relate in particular to prior examinations at different time points in the past. The examination information may relate in particular to pathological changes in the body of the patient, individual measured values, vital-signs data, laboratory values, prior findings, etc. The examination information may be contained as dedicated datasets in the patient data, e.g. in the form of an image dataset or a text file of a medical report. Alternatively or in addition, examination information may be distributed or hidden in the patient data PD. In particular, examination information may relate to information that has been extracted from a dataset contained in the patient data PD (either in the past or within the scope of step S30). For example, the examination information may relate to individual, in particular pathological changes or tissue changes in the body of the patient, such as e.g. lesions in lung, intestines, liver or breast, or tumor-like changes. Such examination information can be found and analyzed automatically or semi-automatically e.g. by automatic image analysis applications. Alternatively or in addition, the examination information may have been generated manually and recorded e.g. in a medical report.

In an optional substep S31, a prioritization or order of precedence of the examination information can be established. This prioritization can specify e.g. how relevant individual pieces of examination information are likely to be to the user. In this case the prioritization may be based in particular on a relative relevance of the pieces of examination information with regard to one another. The prioritization may be e.g. rule-based in that, for example, examination information dating from long in the past is deprioritized or certain types of examination information are regularly given precedence (e.g. image data before laboratory data, or vice versa). Furthermore, a prioritization may be established based on correlation with the decision-making task. If the diagnostic assessment task includes e.g. semantically similar terms to a medical report, this medical report is likely relevant as examination information. Such an analysis can be accomplished e.g. by computational linguistics algorithms.

In the optional substeps S32, S33 and S34, a pictogram PIK can furthermore be assigned to each piece of identified examination information in preparation for the visualization. To that end, attributes are extracted initially in substep S32 in each case from the examination information, which attributes describe one or more properties of the examination information. This may be for example the type of examination information and for instance specify whether the examination is a laboratory examination, a radiological examination, a histopathological examination, a medical report, etc. (cf. FIGS. 3 and 4, windows F4 and F6). In addition, an attribute may relate to an immediate property of a diagnostic finding, such as e.g. the calcium score, the fat content or the contour of a lesion (cf. FIG. 4, windows F1, F2 and F5). In a further substep S33, a predetermined number of pictograms PIK is provided, each of which is associated with one or more typically occurring attributes. In a further substep, each piece of examination information is assigned at least one pictogram PIK based on its respective attributes, which pictogram then allows the corresponding properties of the examination information to be inferred. As shown in FIG. 4, all or selected pictograms PIK can be presented in a key in the user interface (cf. windows F5 and F6) to provide better guidance for the user.

In a further optional substep S35, time points and/or time ranges associated with the examination information are evaluated. In particular, one or more time points and/or time ranges can be selected in substep S35 in order to filter out the examination information corresponding to these time points or time ranges for the further steps. In this case the one or more time points or time ranges can be selected by a user, for example with the aid of a timeline shown in the graphical user interface (cf. FIGS. 3, 4, window F4). Alternatively or in addition, the one or more time points or time ranges may also be selected automatically. In particular, this can be done on the basis of the patient data PD, the examination information and/or the diagnostic assessment task.

In a further optional substep S36, a synopsis is produced for the examination information, which synopsis provides a brief summary of essential properties of the respective examination information. The synopsis may indicate e.g. a type of the examination information, an acquisition or generation time, significant attributes, etc.

In a step S40, an anatomical position within the body model KM is determined for each piece of identified examination information. The anatomical position in the body model KM may be determined for example by registration algorithms. In this case e.g. a registration with the body model may be carried out based on automatically detected landmarks. To that end, information relating to landmarks is first determined in the patient data PD or examination information and the relative position with respect to these landmarks is transferred onto the body model KM. If the underlying examination information contains image data, such landmarks can be detected automatically. An already proposed method is described in U.S. Pat. No. 8,311,303 B2. In addition, however, the patient data PD may also include structured and unstructured text files, e.g. in the form of medical reports. In the case of structured text files (with e.g. separate sections for head, neck/shoulder, thorax, abdomen/pelvis), this structure can be used for determining the anatomical position of individual pieces of information. If this is not possible, the anatomical position of individual pieces of examination information can be determined e.g. by semantic analysis of the examination information, e.g. using a computational linguistics algorithm. In particular, the anatomical position of a piece of examination information can be determined by assigning the examination information to a segment SEG of the body model KM.

In step S40, a visualization VIS is generated for the user based on the examination information, the anatomical positions and the body model KM. Examples of visualizations VIS are shown in FIGS. 3 and 4. The visualization VIS can contain a schematic image of the body model KM in which the anatomical positions of the examination information are highlighted in the body model KM, e.g. by icons IC (see window F1 in FIG. 3). As shown in FIG. 4, the visualization VIS may comprise different views of the body model KM (windows F1 and F2 in FIG. 4). The visualization VIS may be interactive. The interaction with the body model KM may comprise, among other things, an alignment, zooming and/or filtering of the body model KM. Zooming can happen e.g. continuously or by selecting a level of detail DS1, DS2, DS3 (cf. window F3 in FIGS. 3 and 4). Furthermore, filtering of the examination information may also be accomplished using the levels of detail DS1, DS2, DS3. Alternatively and/or in addition, filtering may be carried out according to time points and/or time ranges, e.g. via a timeline shown in the graphical user interface GUI (cf. window F4). Alternatively or in addition, filtering may be carried out according to a type of the examination information, e.g. via clicking on a pictogram in the key (cf. window F6). Alternatively or in addition, the filtering and/or zooming and/or alignment of the body model KM may be performed automatically, in which case in particular the results of steps S21, S22, S31 and/or S35 can be taken into account.

Furthermore, the pictograms PIK optionally determined in steps S32 to S34 may also be shown in the visualization VIS, thus providing an overview of one or more attributes of the corresponding examination information. As shown by way of example in FIG. 4, the pictograms PIK can be shown in particular instead of the icons IC. To provide better guidance, a key to the pictograms can also be shown, by way of which individual attributes can additionally be selected e.g. by mouse click in order thereby to mark or filter one or more pieces of examination information. In addition, the synopsis SYN produced in the optional substep S36 may also be inserted. This can be done either statically (e.g. next to the icon IC or pictogram PIK marking the anatomical position) or dynamically, e.g. in response to a clicking on the anatomical position or icon IC or pictogram PIK or in response to a “hovering” of a pointer (e.g. the mouse pointer) over the anatomical position or icon IC or pictogram PIK (cf. FIG. 3). In addition, selected contents of individual pieces of examination information may also be displayed. As shown in FIG. 3 (cf. window F2), this may be e.g. image data relating to a piece of examination information. The display of such selected contents can be activated e.g. by user selection, for instance by clicking on the corresponding examination information.

Finally, step S60 is directed to the providing of the visualization VIS. This may comprise for example a displaying of the visualization VIS via a graphical user interface GUI and/or a storing of the visualization VIS.

An optional step S70 may then be directed to the generation of a medical report based on the identified examination information. In particular, the results of steps S21, S22, S31 and/or S35 may be taken into account in this case so that the generation of the medical report is based on the automatically and/or manually selected examination information.

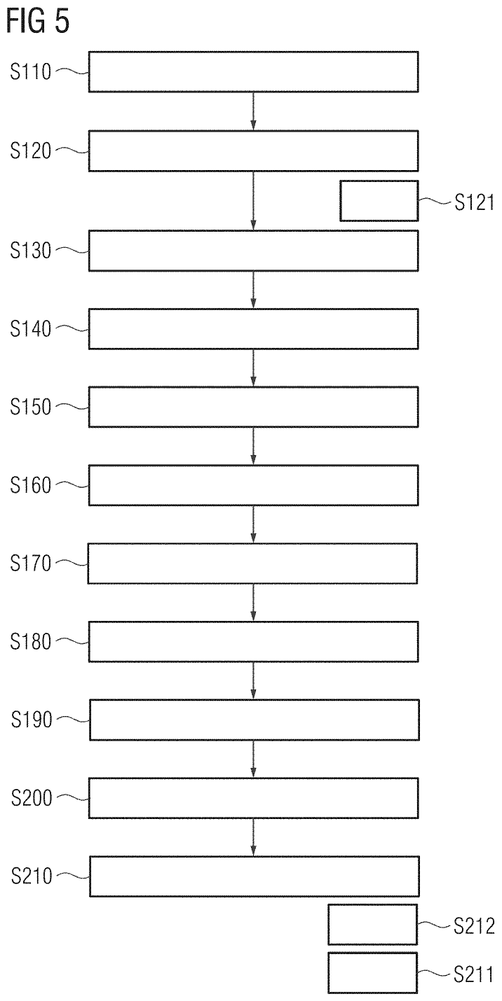

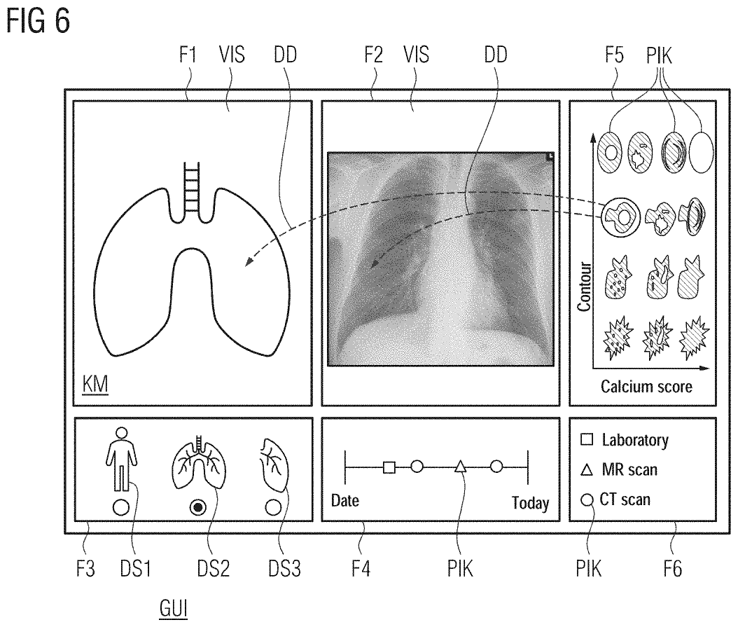

FIG. 5 shows a schematic flowchart of a method for ascertaining examination information during the diagnostic assessment of patient data PD relating to a patient. The order of the method steps is limited neither by the illustrated sequence nor by the chosen numbering scheme. Thus, the order of the steps can be swapped if necessary and individual steps can be omitted. Furthermore, one or more steps, in particular a sequence of steps, and optionally the entire method can be performed repeatedly. FIG. 6 shows an exemplary embodiment of a corresponding graphical user interface GUI by way of example. Elements labeled with the same reference signs denote like or functionally like elements as in FIGS. 4 and 5.

Step S110 is directed to the providing of the patient data PD. Step S110 substantially corresponds to step S10.

Step S120 is directed to a generation of a visualization VIS based on the patient data. In particular, the visualization VIS may be based on a body model KM (cf. FIG. 6). Step S120 may then substantially comprise steps S20 and S50 (including the optional substeps). Alternatively or in addition, the visualization VIS may be based on image data from the patient data PD that represents a part of the body of the patient as a real image. In an optional substep S121, an anatomical region or suitable image data may be selected for the visualization VIS, the visualization VIS then displaying the anatomical region or the corresponding image data. The selection can be made manually by the user or be effected automatically on the basis of the patient data PD, possibly already existing examination information and/or a diagnostic assessment task. As similarly described in connection with FIG. 2, the visualization VIS may be adapted interactively. The visualization VIS is displayed to the user via a user interface 10 in step S130.

In step S140, a predetermined number of pictograms PIK is provided. In this case the pictograms can be embodied as described in connection with step S30 and in particular correspond in each case to one or more attributes relevant to a diagnostic assessment of patient data. For the diagnostic assessment of lesions in a radiological image dataset, the pictograms PIK may correspond for example in each case to different calcium scores, contours, fat contents, etc. (cf. FIG. 6, window F5).

In step S150, at least some of the predetermined number of pictograms PIK are selected. This can be done manually by the user (e.g. by scrolling in the key shown in window F5 in FIG. 6) and/or automatically based on the patient data PD, possibly already existing examination information and/or a diagnostic assessment task. Thus, e.g. a set of pictograms PIK relevant to a diagnostic assessment task may be selected (e.g. pictograms PIK characterizing lung lesions if a corresponding examination of a radiological image of the lung is pending).

In step S160, the set of selected pictograms PIK is then displayed to the user via the user interface 10, e.g. in a graphical user interface GUI (cf. FIG. 6, window F5). The graphical user interface GUI is in this case embodied in particular in such a way that the user is able to select individual pictograms PIK, drag them onto the visualization VIS and drop them there on a drop site (“drag and drop” being the term commonly used for this form of interaction).

Step S170 is directed to a receiving of a user input DD comprising a selecting, dragging and dropping of one of the displayed pictograms PIK onto a drop site in the visualization VIS.

In step S180, an anatomical position is determined based on the drop site. Substantially the same procedure as described in step S40 can be carried out here. The anatomical position may in this case relate to a patient coordinate system (which may be spanned e.g. by one or more anatomical landmarks). For example, the drop site may be registered with the patient coordinate system. Alternatively, the anatomical position may also relate to the body model KM on which the visualization VIS is based or to the image data on which the visualization is based. In step S190, the attributes associated with the selected pictogram PIK are determined in addition.

The attributes and the anatomical position are then merged in step S200 to form examination information which is provided in step S210. The providing may in this case comprise in particular a storing of the examination information in the patient data PD. In an optional substep S220, a medical report may furthermore be produced based on the newly ascertained examination information and where applicable already existing examination information, in which case substantially the same procedure as described in step S70 can be followed.

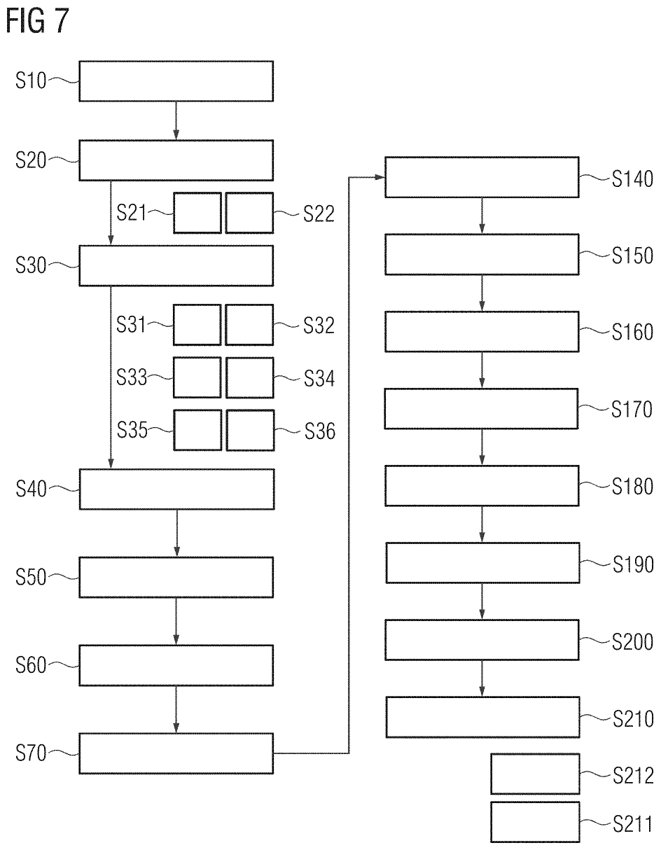

FIG. 7 shows a schematic flowchart of a method for structuring examination information and for ascertaining examination information during the diagnostic assessment of patient data PD relating to a patient. The order of the method steps is limited neither by the illustrated sequence nor by the chosen numbering scheme. Thus, the order of the steps can be swapped if necessary and individual steps can be omitted. Furthermore, one or more steps, in particular a sequence of steps, and optionally the entire method can be performed repeatedly. The flowchart shown in FIG. 7 in this case represents a possible combination of the processes shown in FIG. 2 and FIG. 5. Like reference signs in this case denote like method steps.

Whilst exemplary embodiments have been described in detail in particular with reference to the figures, it should be pointed out that a multiplicity of variations is possible. It should also be pointed out that the exemplary embodiments are merely examples which are in no way intended to limit the scope of protection, the application and the design. Rather, by the foregoing description the person skilled in the art is given a guide for implementing at least one exemplary embodiment, wherein various modifications, in particular alternative or additional features and/or variations of the function and/or arrangement of the described components, may be made as desired by the person skilled in the art without in the process departing from the respective subject matter set forth in the appended claims and its legal equivalent and/or without leaving its scope of protection.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been disclosed in the form of embodiments and variations thereon, it will be understood that numerous additional modifications and variations could be made thereto without departing from the scope of the present invention.

1. A computer-implemented method for structuring medical examination information relating to a patient, the method comprising:
receiving patient data assigned to the patient;
building a schematic body model of the patient based on the patient data, wherein the schematic body model schematically replicates at least one anatomy of the patient;
identifying at least one piece of the examination information in the patient data;
determining an anatomical position for the at least one piece of the examination information within the schematic body model;
generating a visualization of the schematic body model in which the anatomical position of the at least one piece of examination information is highlighted; and
displaying the visualization for a user via a user interface.

2. The method as claimed in claim 1, wherein
the schematic body model is further subdivided into multiple segments, and
the determining the anatomical position includes assigning the at least one piece of examination information to a segment.

3. The method as claimed in claim 2, wherein
the body model is embodied such that each segment is assigned a unique marker, and
the determining the anatomical position determines the anatomical position by identifying at least one of the unique markers for the at least one piece of examination information, the assigning being based on the identifying the at least one of the unique markers.

4. The method as claimed in claim 3, wherein
the unique markers are based on a predetermined anatomical ontology.

5. The method as claimed in claim 2, further comprising:
identifying at least one relevance segment from the segments of the body model based on at least one of the patient data, the at least one piece of examination information or a diagnostic assessment task, and
the generating the visualization includes at least one of highlighting the at least one relevance segment or limiting the visualization to the at least one relevance segment.

6. The method as claimed in claim 5, further comprising:
identifying at least one further piece of examination information in the patient data based on the relevance segment, and
displaying the at least one further piece of examination information via the user interface.

7. The method as claimed in claim 1, wherein a plurality of examination information is identified in the patient data and the method further comprises:
establishing a prioritization of the plurality of examination information based on at least one of the patient data, the examination information or a diagnostic assessment task, wherein the prioritization is based on a relative relevance of a respective piece of examination information within the plurality of examination information, and
the generating the visualization is based on the prioritization.

8. The method as claimed in claim 1, further comprising:
determining one or more attributes based on the at least one piece of examination information;
providing a predetermined number of different pictograms, each pictogram representing different attributes of examination information; and
assigning one pictogram from the number of different pictograms to the at least one piece of examination information based on the determined attributes,
wherein the generating the visualization includes highlighting the anatomical position of the at least one piece of examination information by the assigned pictogram.

9. The method as claimed in claim 1, the method further comprising:
providing a predetermined number of different pictograms, each pictogram representing at least one of different attributes or different attribute combinations of a medical report;
displaying at least some of the predetermined different pictograms for the user via the user interface;
receiving, via the user interface, a user input which comprises the dragging and dropping of a pictogram selected from the displayed pictograms onto a drop site in the displayed visualization;
determining an anatomical position based on the drop site;
determining one or more attributes based on the selected pictogram;
ascertaining a further piece of examination information based on the determined anatomical position and the one or more determined attributes; and
assigning the further piece of examination information to the patient data.

10. The method as claimed claim 1, in which the patient data comprises medical image data which represents an anatomical region of the patient, further comprising:
establishing a registration between the medical image data and the schematic body model;
generating a second visualization based on the medical image data;
displaying the second visualization via the user interface;
receiving a user input via the user interface, the user input is directed to a generation of a further piece of examination information based on the second visualization;
determining an anatomical position for the further piece of examination information based on the user input and the registration;
ascertaining the further piece of examination information based on the determined anatomical position and on the user input; and
assigning the further piece of examination information to the patient data.

11. The method as claimed in claim 1, wherein
the schematic body model comprises a whole-body model of the patient, and
the visualization of the schematic body model comprises a schematic whole-body view of the patient.

12. The method as claimed in claim 1, wherein
the schematic body model includes at least one first level of detail and a second level of detail, wherein the second level of detail is an extract from the first level of detail,
the level of detail is selectable, and
the generating the visualization generates the visualization of the schematic body model based on the selected level of detail.

13. The method as claimed in claim 12, further comprising:
automatically selecting a level of detail based on at least one of the patient data, the at least one piece of examination information or a diagnostic assessment task.

14. The method as claimed in claim 1, wherein
the examination information is associated in each case with at least one time point in the patient trajectory,
at least one of one or more time points or one or more time ranges are selectable, and
the generating the visualization generates the visualization of the schematic body model based on at least one of the selected time points or time ranges.

15. The method as claimed in claim 14, further comprising:
automatically selecting the at least one of one or more time points or one or more time ranges based on the at least one of the patient data, the at least one piece of examination information or a diagnostic assessment task.

16. A computer-implemented method for ascertaining examination information during a diagnostic assessment of patient data relating to a patient, the method comprising:
receiving the patient data relating to the patient;
generating a visualization based on the patient data, the visualization representing at least one anatomical region of the patient;
displaying the visualization for a user via a user interface;
providing a predetermined number of different pictograms, each pictogram representing at least one of different attributes or attribute combinations of a possible medical report of the patient;
displaying at least some of the predetermined different pictograms to allow selection, dragging and dropping of individual pictograms by the user via the user interface;
receiving a user input, the user input comprises a dragging and dropping of a pictogram selected from the displayed pictograms onto a drop site in the displayed visualization;
determining an anatomical position of medical findings based on the drop site;
determining one or more attributes of the medical findings based on the selected pictogram;
ascertaining the examination information based on the determined anatomical position and the one or more determined attributes; and
providing the examination information.

17. The method as claimed in claim 16, wherein the providing the examination information includes at least one of:
producing a medical report based on the examination information, or
storing the examination information in the patient data.

18. The method as claimed in claim 16, wherein
the generating the visualization includes building a schematic body model of the patient, the body model schematically replicates at least one anatomy of the patient, and
the visualization comprises a visual representation of the schematic body model.

19. The method as claimed in claim 16, wherein
the patient data comprises medical image data representing the at least one anatomical region of the patient, and the generating the visualization generates a visualization of the medical image data, the method further comprising:
building a schematic body model of the patient based on the patient data, the body model schematically replicates at least one anatomy of the patient; and
establishing a registration between the medical image data and the schematic body model, wherein
the anatomical position is determined based on the registration, and
the anatomical position is defined relative to the body model.

20. The method as claimed in claim 16, wherein
the generating the visualization further comprises:
selecting the at least one anatomical region of the patient for the visualization,
the visualization represents only the selected anatomical region, and
the selection is made based on at least one of the patient data or a diagnostic assessment task.

21. A computer-implemented method for ascertaining examination information during a diagnostic assessment of patient data relating to a patient, comprising:
receiving the patient data relating to the patient, wherein the patient data comprises medical image data that represents an anatomical region of the patient;
building a schematic body model of the patient based on the patient data, the body model schematically replicates at least one anatomy of the patient;
establishing a registration between the medical image data and the schematic body model;
generating a visualization of the medical image data;
displaying the visualization for a user via a user interface;
receiving a user input via the user interface, the user input is directed to a generation of examination information on the basis of the visualization;
determining an anatomical position for the examination information within the schematic body model based on the user input and the registration;
ascertaining the examination information based on the determined anatomical position and on the user input; and
providing the examination information.

22. A system for structuring medical examination information relating to a patient, the system comprising:
an interface; and
a controller, the controller is configured to cause the system to,
receive patient data assigned to the patient via the interface,
build a schematic body model of the patient based on the patient data, wherein the schematic body model schematically replicates at least one anatomy of the patient,
identify at least one piece of the examination information based on the patient data,
determine an anatomical position for the at least one piece of examination information within the schematic body model, and
generate a visualization of the schematic body model in which the anatomical position of the at least one piece of examination information is highlighted.

23. A system for ascertaining examination information during the diagnostic assessment of patient data, the system comprising:
an interface; and
a controller, the controller is configured to cause the system to,
receive the patient data via the interface,
generate a visualization based on the patient data and to provide it to a user via the interface, the visualization represents at least one anatomical region of the patient,
provide a predetermined number of different pictograms, each pictogram representing at least one of different attributes or attribute combinations of a possible medical report of the patient,
provide a user with at least some of the predetermined different pictograms via the interface to allow selection, dragging and dropping of individual pictograms onto the visualization by the user,
receive a user input of the user via the interface, the user input comprises the dragging and dropping of a pictogram selected from the displayed pictograms onto a drop site in the visualization,
determine an anatomical position based on the drop site,
determine one or more attributes based on the selected pictogram,
determine examination information based on the determined anatomical position and the one or more determined attributes, and
provide the examination information.

24. A system for ascertaining examination information during the diagnostic assessment of patient data, the system comprising:
an interface; and
a controller, wherein the patient data comprises medical image data that represents an anatomical region of the patient, and the controller is configured to cause the system to,
receive the patient data via the interface,
generate a visualization of the medical image data and provide it to a user via the interface,
build a schematic body model of the patient based on the patient data, the schematic body model schematically replicates at least one anatomy of the patient,
establish a registration between the medical image data and the schematic body model,
receive a user input via the user interface, the user input is directed to a generation of the examination information based on the visualization,
determine an anatomical position for the examination information within the schematic body model based on the user input and the registration,
ascertain the examination information based on the determined anatomical position and on the user input, and
provide the examination information.

25. A computer program product which comprises a program, when executed by a programmable computing unit of a system, causes the system to perform the method of claim 1.

26. A computer-readable storage medium having readable and executable program sections, when executed by a controller of a system, cause the system to perform the method of claim 1.
